(19) United States
(12) Patent Application Publication
(10) Pub. No.: US 2023/0310057 A1
(43) Pub. Date: Oct. 5, 2023

(54) SURGICAL CAUTERY ELECTRODE AND HANDPIECE WITH INTEGRAL SMOKE EVACUATION LUMEN

(71) Applicant: David Louis Kirschman, Dayton, OH (US)

(72) Inventor: David Louis Kirschman, Dayton, OH (US)

(73) Assignee: Aerobiotix, LLC, Miamisburg, OH (US)

(21) Appl. No.: 18/180,396

(22) Filed: Mar. 8, 2023

Related U.S. Application Data
(60) Provisional application No. 63/320,282, filed on Mar. 16, 2022.

(51) Int. Cl. A61B 18/08

(52) U.S. Cl. CPC A61B 18/082; A61B 2018/00595

(57) ABSTRACT

This invention relates to a cautery instrument or handheld device having a generally centralized lumen or passageway through the device and that utilizes a conductive tubular member.

### CROSS-REFERENCE TO RELATED APPLICATION

The present application claims priority to provisional U.S. Application Serial No. 63/320,282 filed Mar. 16, 2022, to which Applicant claims the benefit of the filing date of this provisional application, which is incorporated herein by reference and made a part hereof.

### BACKGROUND OF THE INVENTION

### 1. Field of the Invention

This invention relates to a cautery instrument or handheld device having a generally centralized lumen or passageway through the device and that utilizes a conductive tubular member.

### 2. Description of the Related Art

The use of electrocautery is commonplace in surgical procedures for tissue dissection and hemostasis. The cautery process involves the controlled application of electrical current onto biological tissue. This results in a significant smoke plume of toxic gases, such as benzene, toluene, ethyl benzene, xylene, styrene, formaldehyde, and acetaldehyde, with significant occupational exposure of surgical personnel.

The basic surgical tool used in the cauterization process is the monopolar electrocautery handpiece. This handpiece typically consists of a wire lead, body, finger switch, and electrode. Electrodes can be available in various sizes and dimensions and are made of conductive material. The most common electrode shape is a linear elongated or flattened form. In order to reduce the emission of smoke plume from the cautery handpiece, several firms have incorporated a vacuum means to the handpiece, where there is a vacuum opening in the vicinity of the electrode. This opening typically transmits through the handpiece to a flexible vacuum tube and external vacuum source.

Referring now to FIGS. 1A-1B, a prior art cauterizing system and electrode is shown. The device has an electrode A with the opening of separate external evacuation tube B and an actuation button known in the prior art. The cautery device of the prior art had a non-central and non-linear evacuation tube B that was coupled to a vacuum connection E that coupled the device to a vacuum source VS. Note that the surgeon would typically hold both the handpiece body and the external evacuation tube during a cautery procedure.

In the prior art, the vacuum opening has generally been positioned in two ways, either surrounding the electrode, or adjacent to the electrode. Each of these approaches has significant disadvantages. The first disadvantage is visual obstruction, wherein the vacuum opening comprises a tube obstructing the surgeon’s vision of the electrode tip. A second disadvantage is the distance of the vacuum opening from the electrode tip, wherein smoke plume can escape between its point of generation at the electrode tip and the evacuation opening. A third disadvantage is bulk. Compared to non-evacuating electrosurgical handpieces, the evacuating handpieces are significantly larger. This is due to the need for evacuation air to run outside of the electrode and eccentrically in the handpiece body.

What is needed, therefore, is a system, method and means for providing a cautery instrument that overcomes one or more of the disadvantages of the prior art.

### SUMMARY OF THE INVENTION

Therefore, it is the object of the current invention to provide an electrocautery electrode design and handpiece which addresses these and more shortcomings of the prior art. The current invention provides a conductive electrode with a tubular body and central lumen for plume evacuation directly through the electrode, further comprising a substantially flat and/or linear extension. The invention further describes a handpiece design which allows for a hollow electrode to be energized and attached to an external vacuum source. This design has the benefits of improved surgical visualization, reduced bulk, and improved evacuation performance compared to the prior art.

One object of the invention is to provide a conductive tubular member that provides a conductive path for current as well as defines a centralized lumen through the device.

Another object of the invention is to provide a cautery device that is adapted to deliver current to a cautery tip which may be curved, planar, non-planar, or the like.

Another object of the invention is to provide a conductive tubular member that defines a passageway or lumen for evacuating smoke and vapor from a surgical site.

Still another object of the invention is to provide a cautery instrument that is not as bulky as the instruments of the prior art.

Yet another object of the invention is to provide a cautery instrument that is adapted to be utilized with bipolar and unipolar methods of operation.

Still another object of the invention is to provide is to provide a cautery tip that has a thickness that is generally similar to the thickness of the tubular wall that defines the cautery instrument.

Yet another object of the invention is to provide a cautery tip that is integrally or monolithically formed from a tubular wall which defines a lumen or passageway in the instrument.

In one aspect, one embodiment of the invention comprises an electrocautery instrument for cauterizing a surgical site associated with a patient, the electrocautery instrument comprising a tubular body having a passageway for evacuating vapor from the surgical site, the tubular body comprising an extension for cauterizing at the surgical site; the extension being conductive and adapted to be coupled to a power source for energizing the extension; the tubular body having an inlet end having the extension and an outlet end coupled to a vacuum source adapted to create a predetermined pressure in the passageway in order to evacuate vapor from the surgical site through the tubular body prior to, during or after use of the electrocautery instrument at the surgical site, the vapor comprising at least one of smoke, cauterizing vapor, gas, liquid or debris; and wherein the passageway is defined by the tubular body and causes the vapor resulting from the cauterizing at the surgical site to be evacuated through the tubular body.

In another aspect, another embodiment of the invention comprises an electrocautery handpiece which comprises a tubular handpiece body; an internal airflow means; a vacuum connection; a substantially hollow tubular electrode communicating with the airflow means; the electrode comprising an integral, substantially elongated extension from the tubular handpiece body, wherein electrical current is transmitted from the tubular handpiece body to the extension, and surgical smoke enters the tubular handpiece body and is evacuated via the internal airflow means.

In yet another aspect, another embodiment of the invention comprises an electrocautery electrode for the transmission of electrical current and surgical smoke plume, the electrode comprising a substantially tubular portion with at least one integral substantially linear extension; the extension integral with a portion of the tube wall.

In still another aspect, another embodiment of the invention comprises a method for the evacuation of surgical cautery smoke comprising generation of smoke plume from a substantially linear and/or flattened electrode tip, the tip being in integral communication with a tubular electrode body, the smoke flowing through a tubular electrode body in communication with an electrocautery handpiece comprising a substantially central lumen, the lumen being in communication with a vacuum source via a vacuum connection means to evacuate the smoke.

This invention, including all embodiments shown and described herein, could be used alone or together and/or in combination with one or more of the features covered by one or more of the following list of features:
The electrocautery instrument wherein the tubular body is conductive and creates a conductive path for current to flow from a power source to the extension.
The electrocautery instrument wherein the extension is integral with the tubular body and both are conductive.
The electrocautery instrument wherein the tubular body is defined by a wall having at least one predetermined dimension, the wall defining the passageway and the extension.
The electrocautery instrument wherein the extension comprising a cross-sectional dimension that is smaller than the at least one predetermined dimension.
The electrocautery instrument wherein an elongated portion of the extension comprises a first surface and a generally opposing second surface, the first surface defining a working surface having a generally curved or arcuate portion.
The electrocautery instrument wherein the working surface has a radius of curvature substantially approximating a radius of curvature of the wall.
The electrocautery instrument wherein the extension comprises a working surface that is generally planar.
The electrocautery instrument wherein the extension is detachably secured to the tubular body with a predetermined connection, the tubular body and the extension both being conductive to permit the extension to cauterize at the surgical site.
The electrocautery instrument wherein the predetermined connection is a least one of a threaded, press-fit, bayonet, or socket connection.
The electrocautery instrument wherein the passageway is substantially centrally located in the tubular body to define a substantially linear or non-serpentine conduit for evacuating the vapor.
The electrocautery instrument wherein the tubular body comprises an interior wall that defines the passageway, at least a portion of the interior wall having a catalyst coating, a single uncoated material or other forms of deposition, such as plasma spraying and electroplating.
The electrocautery instrument wherein the passageway is substantially centrally located in the tubular body to define a substantially linear or non-serpentine conduit for evacuating the vapor.
The electrocautery instrument wherein extension comprises an elongated portion having a predetermined shape adapted to cauterize at the surgical site.
The electrocautery instrument wherein the tubular body comprises an interior wall that defines the passageway to have a primary flow path through the tubular instrument, the extension lying lateral to the primary flow path.
The electrocautery instrument wherein the tubular body comprises an interior wall that defines the passageway to have a primary flow path through the tubular instrument, the extension lying substantially in the primary flow path so that the vapor flows past at least a portion of the extension before it enters the passageway for evacuation.
The electrocautery instrument wherein the extension comprises an extension axis that is generally coaxial with an axis of at least one of the tubular body or the passageway.
The electrocautery instrument wherein the extension comprises an extension axis that is generally coaxial with an axis of at least one of the tubular body or the passageway.
The electrocautery instrument wherein the extension comprises a first portion integrally formed with a first portion of the wall and having a first surface for performing work and a second surface for facilitating directing the vapor into the passageway.
The electrocautery instrument wherein the extension comprises a first portion integrally formed in a first area of the wall and a second portion integrally formed in a second area of the wall, the extension comprising a first surface and generally opposed second surface, both of which are in an airflow path defined by the passageway.
The electrocautery instrument wherein the first area and the second area are each associated with an inlet end of the tubular body, the extension comprising a cauterizing tip situated substantially between the first and second portions so that the first and second portions support the first and second surfaces in the airflow path to permit vapor to flow past the first and second surfaces and into the passageway.
The electrocautery instrument wherein the extension is configured or adapted to facilitate directing and evacuating vapor into the passageway during a cauterizing procedure.
The electrocautery instrument wherein an elongated portion of the extension comprises a first surface and a generally opposing second surface, the first surface having a generally curved or arcuate portion.
The electrocautery instrument wherein an elongated portion of the extension comprises a first surface and a generally opposing second surface, the first surface defining a working surface for engaging a patient and having a generally curved or arcuate portion.
The electrocautery instrument wherein the first surface is generally curved and has a radius of curvature approximating a radius of curvature of an outer surface of the tubular body, the second generally opposing surface having a predetermined cross-sectional shape that is different from the first surface.
The electrocautery instrument wherein the first surface is generally curved and the second surface is generally planar.
The electrocautery instrument wherein an elongated portion of the extension comprises a first surface and a generally opposing second surface, the first and the second surfaces both being generally planar.
The electrocautery instrument wherein the tubular body comprises an insulator.
The electrocautery instrument wherein the tubular body comprises a non-conductive handpiece body for gripping the tubular body.
The electrocautery instrument wherein the extension extends from an inlet end of the tubular body, the inlet end having an end wall portion that defines an inlet opening sized and adapted to receive vapor from the surgical site.
The electrocautery instrument wherein the end wall portion lies in a predetermined plane relative to an axis of the tubular body, the end wall portion of tubular body and the extension cooperating to define a predetermined angle of approximately 90 degrees or is acute.
The electrocautery instrument wherein the end wall portion lies in a predetermined plane relative to an axis of the tubular body, the end wall portion defines an angled tubular body wall that lies in a plane relative to the axis of the tubular body that is obtuse.
The electrocautery instrument wherein the tubular body comprises a working end having an end wall that lies in an imaginary plane that intersects an axis of the passageway at a predetermined angle.
The electrocautery instrument wherein the predetermined angle is approximately 90 degrees.
The electrocautery instrument wherein the predetermined angle is an angle other than 90 degrees.
The electrocautery instrument wherein the extension is generally elongated and planar.
The electrocautery instrument as recited in claim 1 wherein the extension is generally non-planar.
The electrocautery instrument wherein the extension is stepped and comprises a first surface that extends from a wall of the tubular body and a second surface, the first and second surfaces lying in different planes.
The electrocautery instrument wherein the first and second surfaces are coupled by a joining portion, the first surface, the second surface and the joining portion being configured such that the second surface is situated substantially in an airflow path defined by the passageway.
The electrocautery instrument wherein the second surface defines a cauterizing surface that is substantially centrally located in the airflow path, the airflow path through the tubular body being substantially linear.
The electrocautery handpiece wherein the surgical smoke arises from the electrode and is evacuated through the internal airflow means to the vacuum connection in a substantially linear manner.
The electrocautery handpiece wherein the internal airflow means comprises a channel located in the substantial center of an axis of the tubular handpiece body and is substantially linear.
The electrocautery handpiece wherein an evacuation airflow pathway travels in a substantially linear manner from its point of generation at the electrode to a vacuum connection means at a base of the handpiece.
The electrocautery handpiece wherein the electrocautery handpiece comprises the vacuum connection comprising a barbed fitting, threaded fitting, bonded fitting, swivel fitting, valve fitting or similar to allow for connection of the handpiece to a vacuum source via flexible surgical tube or similar implement.
The electrocautery handpiece wherein the electrocautery handpiece comprises an electrical supply and/or switching means for the provision of cauterizing current to the electrode, the current delivered to substantially tubular portion of the electrode for transmission to the extension.
The electrocautery electrode wherein the integral extension thickness is less than the tubular wall thickness with at least one flattened surface opposing the curved surface of the tubular wall.
The electrocautery electrode wherein a portion of the electrode and/or the extension is electrically insulated with a non-conductive coating, covering or material.

These and other objects and advantages of the invention will be apparent from the following description, the accompanying drawings and the appended claims.

### BRIEF DESCRIPTION OF THE ACCOMPANYING DRAWINGS

FIGS. 1A-1B illustrate a prior art cautery device having an external lumen;

FIGS. 2A-2J illustrate one embodiment of the invention having a cautery tip having a curved and generally planar upper surface, wherein FIG. 2B is a sectional view taken along the line 2B-2B in FIG. 2H;

FIGS. 3A-3I illustrate another embodiment of the invention having a cautery tip that is generally planar on upper and lower surfaces, wherein FIG. 3B is a sectional view taken along the line 3B-3B in FIG. 3H;

FIGS. 4A-4I illustrate still another embodiment of the invention having a cautery tip with a lower curved surface and generally planar upper surface and also illustrating an angled end wall to provide more relief, wherein FIG. 4B is a sectional view taken along the line 4B-4B in FIG. 4H;

FIGS. 5A-5I illustrate another embodiment of the invention, wherein FIG. 5B is a sectional view taken along the line 5B-5B in FIG. 5H;

FIGS. 6A-6I illustrate another embodiment of the invention with a stepped cautery tip, wherein FIG. 6B is a sectional view taken along the line 6B-6B in FIG. 6H;

FIGS. 7A-7H illustrate an embodiment of the invention with a cautery tip having curved upper and lower surfaces;

FIGS. 8A-8J illustrate an embodiment of the invention showing a cautery tip that lies in a generally vertical plane;

FIGS. 9A-9E illustrate various embodiments of a plurality of cautery tips, each having different predetermined shapes;

FIGS. 10A-10B illustrate an embodiment of the invention with a monopolar method of operating and a bipolar method of operation that may be utilized with one or more of the embodiments described herein;

FIGS. 11-27 are various views showing further details of an ornamental design of the tapered cautery tip shown in FIGS. 4A-4I; and

FIGS. 28-44 are various views showing further details of an ornamental design of the bayonet cautery tip shown in FIGS. 6A-6I.

### DETAILED DESCRIPTION OF THE PREFERRED EMBODIMENTS

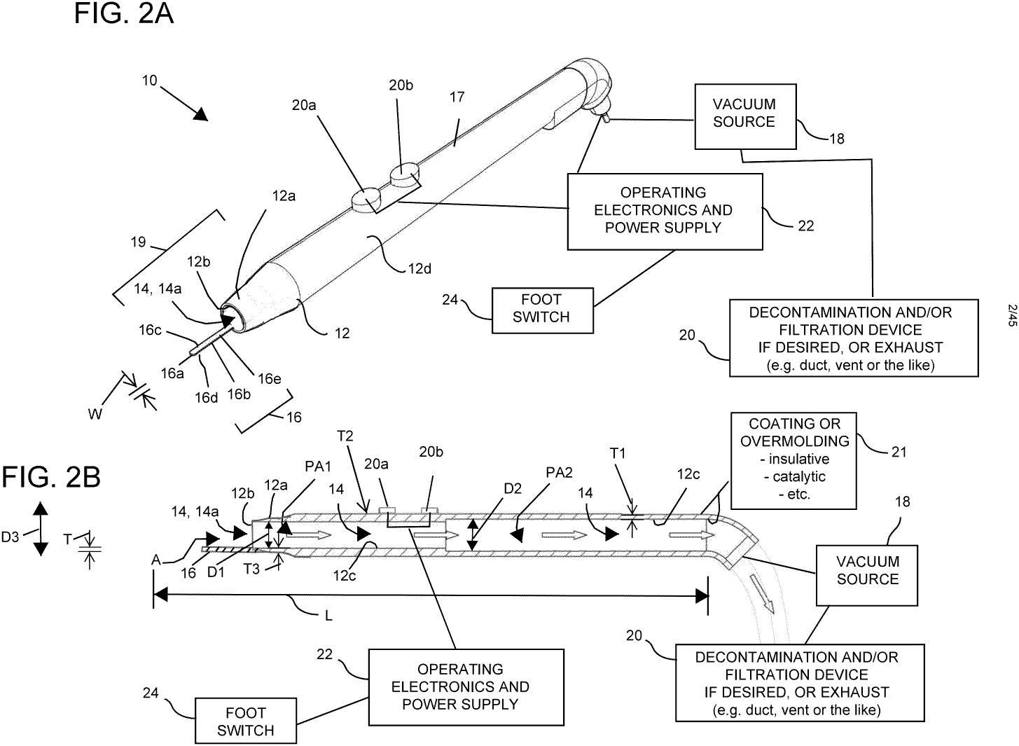

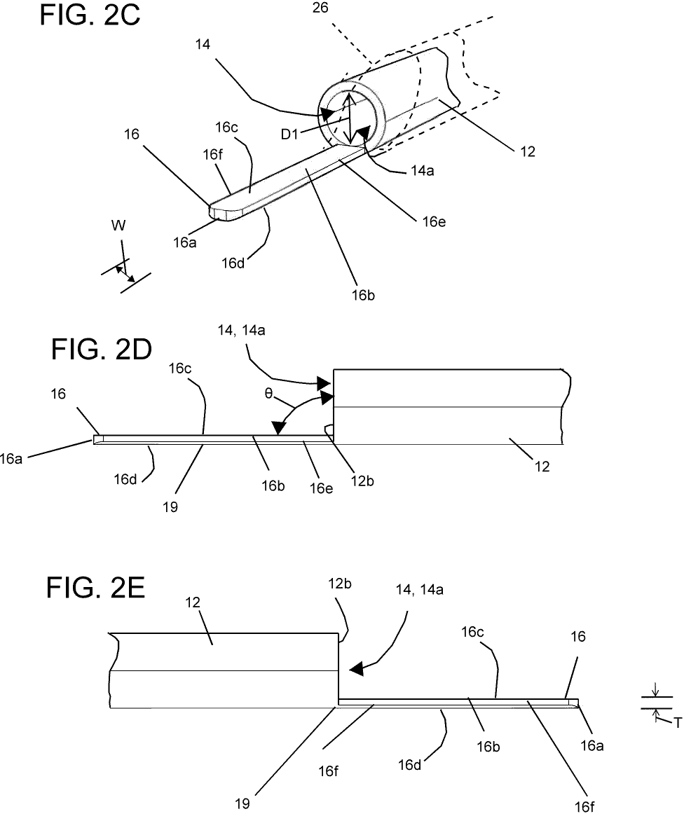

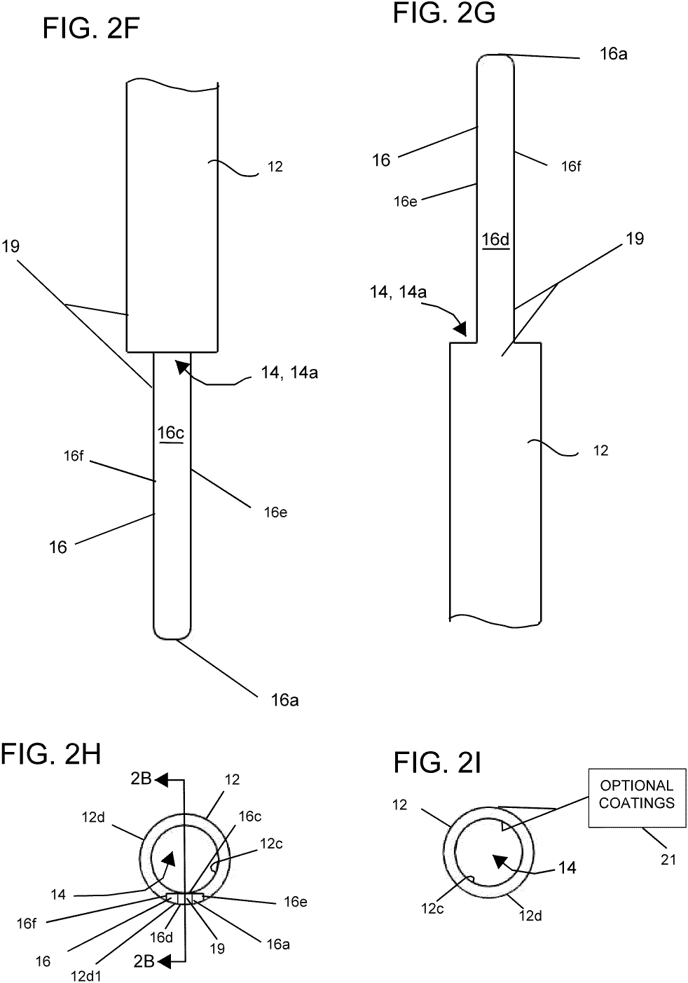

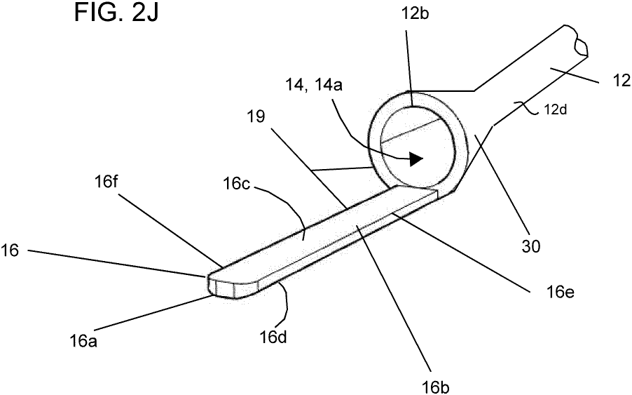

Referring now to FIGS. 2A-10B, an electrocautery instrument and system 10 is shown. The electrocautery instrument and system 10 is adapted to provide a tubular body 12 that is adapted to provide a cautery electrode and handpiece with an integral and generally centrally located smoke and vapor evacuation passageway or lumen 14 which will be described in more detail later herein. The electrocautery instrument and system 10 is adapted for cauterizing or cutting a surgical site of a patient.

Referring now to FIGS. 2A-2B, a first embodiment is shown. The electrocautery instrument and system 10 comprises the tubular body 12 that may be constructed of a substantially electrically conductive material, such as steel, iron, aluminum, titanium, tantalum, platinum, gold, palladium, manganese, either in singular construction or utilizing various combinations of coatings, depositions, or composites. The tubular body 12 may be a one-piece monolithic construction, a multi-piece construction or in singular construction for utilizing the various combinations method. The tubular body 12 is conductive and provides an electrical connection and conductive path between an operating electronics and power supply 22 and a cautery tip 16. Note that the generally centralized passageway or lumen 14 is adapted for evacuating smoke or vapor from the surgical site during a surgical procedure. The tubular body 12 comprises a cautery extension 19, which comprises a tubular end wall portion 12a and the cautery tip 16. Thus, note that the cautery extension 19 comprises both the generally circular tubular end wall portion 12a and the cautery tip 16 for cauterizing. The cautery tip 16 is monolithically or integrally formed as part of the tubular body 12 and is conductive and permits current to pass therethrough and to the cautery tip 16.

The tubular end wall portion 12a comprises an end wall 12b of the tubular body 12 and the cautery tip 16, both of which are conductive and integral with or monolithically formed as part of the tubular body 12. Thus, it should be appreciated that the tubular body 12, the cautery extension 19, including the tubular end wall portion 12a and the cautery tip 16, are all integral, monolithically formed or conductively coupled so that electrical current can flow therethrough and the cautery tip 16 can cauterize the surgical site.

In the illustration being described, the tubular body 12 comprises a diameter of approximately 0.100 to 0.400 inch. As illustrated in FIG. 2B, the tubular body 12 comprises an inner wall 12c that defines a diameter D1 of about 0.004 to .125 inch, which also defines an inlet 14a of the passageway or lumen 14. It should be understood that these diameters could be smaller or larger if desired. A coating, described later herein, may also be applied to the inner wall 12c.

The electrocautery instrument and system 10 comprises an external outer wall 12d having a diameter D3 and that defines a grip or gripping portion 17 for enabling a surgeon or person to grip the electrocautery instrument and system 10. It is to be noted that the gripping portion 17 is defined by the outer wall 12d of the tubular body 12. Although not shown, it may also have an insulative coating, sleeve or overmolding if desired.

It should be appreciated the inner and outer diameters D1 and D3 of the tubular body 12 may be constant over length L of the electrocautery instrument and system 10 or, alternatively, one or both diameters of the inner wall 12c and outer wall 12d may vary over the length L as illustrated in FIG. 2B by diameter D2. The outer wall 12d may also be formed or machined to provide unique gripping features for facilitating gripping the electrocautery instrument and system 10.

In the embodiment of FIGS. 2A-2J, it should be understood that the cautery extension 19 has a general overall length of about 0.250 to 1.250 inch and a width of about 0.020 to 0.160 inch. In the embodiment being described, the cautery tip 16 comprises a tip thickness T (FIG. 2B) that is about 0.040 to .120 inch and a width W (FIG. 2C) that is about 0.040 to .200 inch. It should be understood that these dimensions may be smaller or larger if desired.

In the illustration being described, it should be appreciated that the inner wall 12c may comprise the diameter D1 that is continuous throughout at least a portion or all of the length L (FIG. 2B) of the tubular body 12. Alternatively, the inner wall 12c diameter may be stepped (e.g., from D1 to D2) or may change over the length L. For example, in the illustration shown in FIG. 2B, the tubular body 12 may comprise a first diameter D1 (FIG. 2B) near the inlet 14a that either gradually increases throughout the length L of the tubular body 12, or as illustrated in FIG. 2B, the diameter can be stepped or change, for example, to a larger second diameter D2 that is sized and dimensioned to facilitate increasing a negative pressure in an area A at the inlet 14a of the passageway or lumen 14.

In the illustration being described, the inner wall 12c defines a first pressure area PA1 (FIG. 2B) and a second pressure area PA2 which is generally larger than the pressure area PA1, both of which facilitate evacuating smoke or vapors resulting from the cautery process at the cautery tip 16. In this regard, the electrocautery instrument and system 10 comprises a vacuum source 18 for creating negative pressures at the pressure areas PA1 and PA2 and also for creating a pressure differential between the ambient atmosphere outside the tubular body 12 and the pressure areas PA1 and PA2 in order to effectively cause the smoke and vapor to be evacuated or vacuumed into the inlet 14a of the passageway or lumen 14 and be collected by the vacuum source 18.

The vacuum source 18 may be coupled to a decontamination and/or filtration device 20 that collects the vacuumed smoke and vapor and decontaminates and/or filters it so that it can be returned to the environment. One such device is the ILLUVIA® system available from Aerobiotix, LLC located in Miamisburg, Ohio. Alternatively, the vacuum source 18 may be coupled directly to an external outlet, such as a duct system (not shown), where the evacuated smoke and vapors can be exhausted from the surgery room.

As previously mentioned, the tubular body 12 and the cautery extension 19 are integral and conductive or conductively coupled, and again, note that the conductive tubular body 12 and inner wall 12c define the passageway or lumen 14 which is generally centrally through the electrocautery instrument and system 10. Although not shown, it should be understood that the surgeon or person using the electrocautery instrument and system 10 is typically wearing rubber surgical gloves, which insulate the surgeon to prevent a short or a pathway for the current to pass through the tubular body 12.

As previously mentioned, the tubular body 12 may be coated with a coating 21 (schematically illustrated in FIG. 2B). For example, the outer wall 12d may be coated with the coating 21 to facilitate the surgeon’s gripping the electrocautery instrument and system 10. The coating 21 may comprise a sleeve 26 (FIG. 2C) which is either placed on the tubular body 12 or an overmolding applied on the outer wall 12c. Such coating 21 may be adapted to provide finger grip positions onto the outer wall 12c of the tubular body 12 if desired.

Regarding the inner wall 12c, it too may also be coated or treated with the coating 21 that is adapted to treat the smoke or vapor as it passes through the passageway or lumen 14. In this regard, the coating 21 on the inner wall 12c (FIG. 2I), may comprise, for example, a catalytic coating comprising materials which have oxidative catalyst properties, including elements or compounds containing platinum, palladium, manganese, copper, zirconium, redox-active oxides of iron, vanadium, and/or molybdenum, which the smoke and vapor is exposed to as it passes through the passageway or lumen 14. In the illustration being described, the coating 21 is adapted for treating and/or oxidizing the smoke or vapor to a less harmful gas or vapor. The coating 21 may comprise, for example, a titanium dioxide, manganese, zirconium, elements or compounds containing platinum, palladium, copper, redox-active oxides of iron, vanadium, and/or molybdenum.

Advantageously, note in FIG. 2B that the passageway or lumen 14 is generally centralized and provides a direct path for the vapor or smoke to pass through the tubular body 12 and electrocautery instrument and system 10 itself. Thus, the passageway or lumen 14 is generally centrally located with the inlet 14a being proximately located to the cautery tip 16 and near where the cautery process is performed. Because the passageway or lumen 14 is through the tubular body 12, an uninterrupted direct and non-circuitous pathway is provided for evacuating the smoke and vapor through the hand-held tubular body 12 itself and directly to the vacuum source 18. Advantageously, the passageway or lumen 14 is substantially centrally located in the electrocautery instrument and system 10 and provides a substantially central evacuation pathway from the cautery tip 16 to the vacuum source 18. This has been found to substantially improve the evacuation of the smoke and vapor from the surgical site of the patient over the prior art cauterizing devices. Although not shown, the passageway or lumen 14 could be offset from a center axis of the tubular body 12.

The inner wall 12c of the tubular body 12 may comprise a substantially constant thickness or it may have different thicknesses along its length L. In the illustration being described relative to FIGS. 2A and 2B, note that the dimension of the inner wall 12c changes along the length L and has multiple thicknesses, such as thickness T1, thickness T2 and thickness T3. Note that the thickness T3 of the inner wall 12c is substantially the same as and defines the thickness T of the cautery tip 16 prior to further processing or machining, so that at least a portion of the inner wall 12c itself defines the monolithic or integral cautery extension 19, as well as the cautery tip 16. In the illustration of FIGS. 2A and 2B, a portion of the inner wall 12c has been removed by conventional machining techniques to define the cautery extension 19 comprising the inner wall 12c. The removed portion is illustrated in phantom in FIG. 2C.

The electrocautery instrument and system 10 comprise an on/off activation switch 20a and a power level switch 20b. As conventionally known, one of the switches 20a, 20b may be for a high-frequency current while the other switch 20a, 20b may be for a low frequency current which have different cutting/coagulation characteristics. In one alternative embodiment, one of the switches 20a, 20b may be used to control the suction of the electrocautery instrument and system 10. The switches 20a and 20b are coupled to the operating electronics and a power supply 22 that is conventionally known. It is important to remember that the tubular body 12 is conductive and permits current to travel from the operating electronics and power supply 22 through the tubular body 12 and ultimately to the cautery tip to perform the tissue cutting or cauterization. In the illustration being described, the operating electronics and power supply 22 are conventional and available from Aerobiotix, LLC located in Miamisburg, Ohio.

It should be understood that the electrosurgical generator, electronics, and operating characteristics are conventional devices well known to artisans in the field. At a basic level it consists of a power supply, a controller, an AC current generator, a patient grounding pad, and a connection means for an electrosurgical handpiece with an electrode that contacts the patient. When activated, it creates a closed AC circuit with the patient acting as a resistor in the circuit. The switches on the electrocautery instrument and system 10 provide settings of the AC waveform amplitude and frequency. Once these are set, the current is activated by the switches 20a, 20b on the handpiece and/or the foot pedal 24.

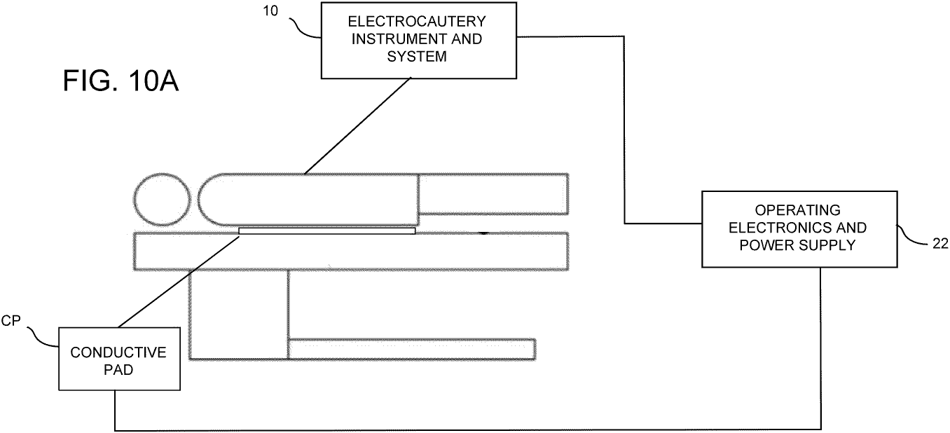

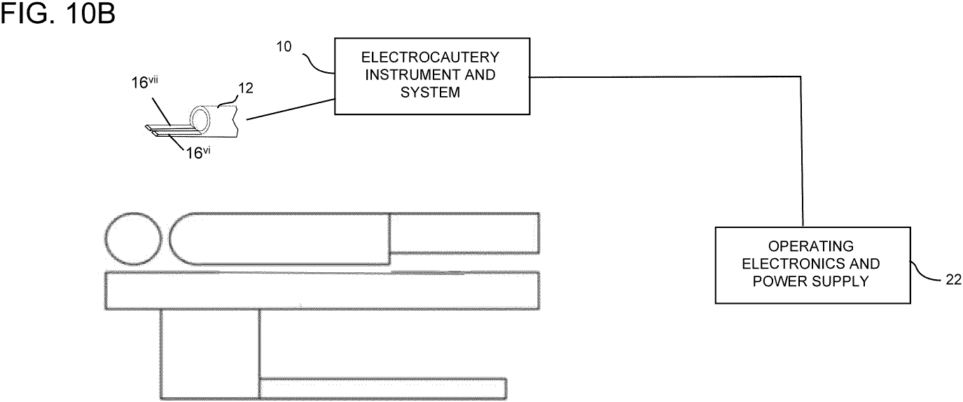

The conductive tubular body 12 defines the pathway between the operating electronics and power supply 22 and the switches 20a and 20b. Again, the tubular body 12 also defines the centralized passageway or lumen 14 for all dimensions of the electrocautery instrument and system 10 which are reduced compared to the devices of the prior art. The conductive tubular body 12 allows current to flow through the tubular body 12 and into the cautery tip 16 for the cauterizing process. The electrocautery instrument and system 10 may utilize either a polar or bipolar process as is conventionally known and as described later herein relative to FIGS. 10A and 10B.

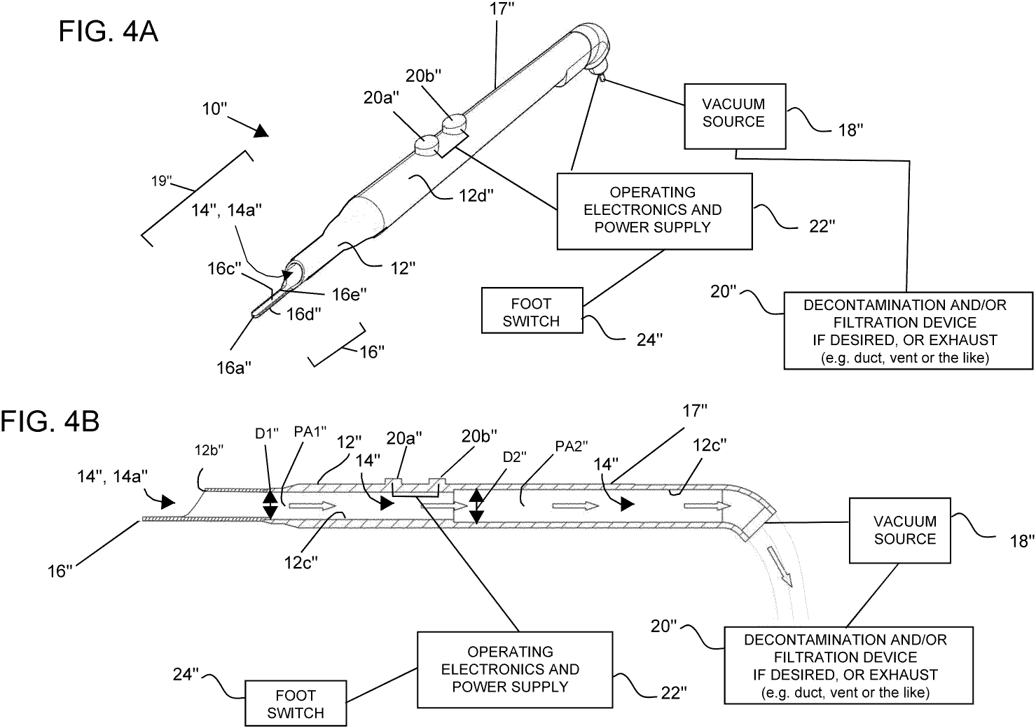

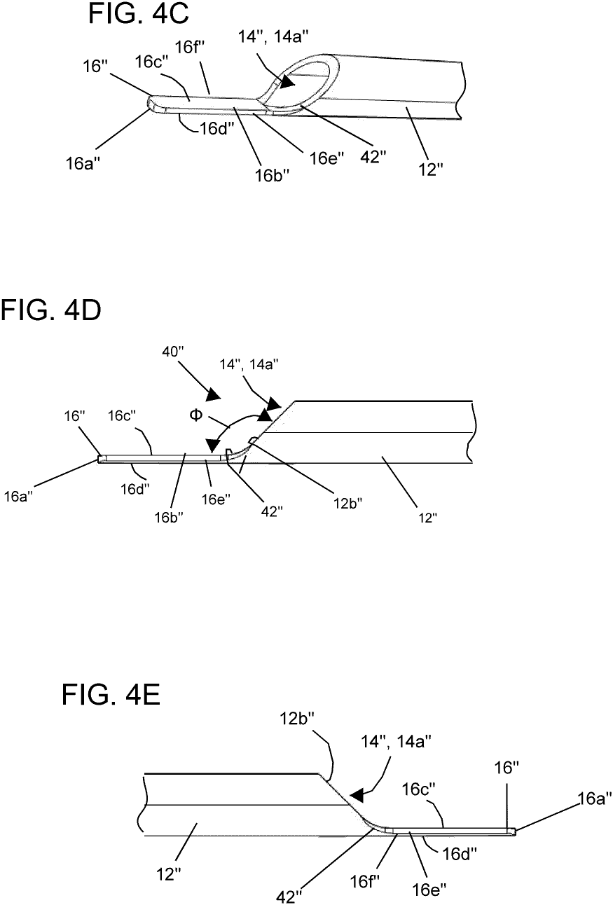

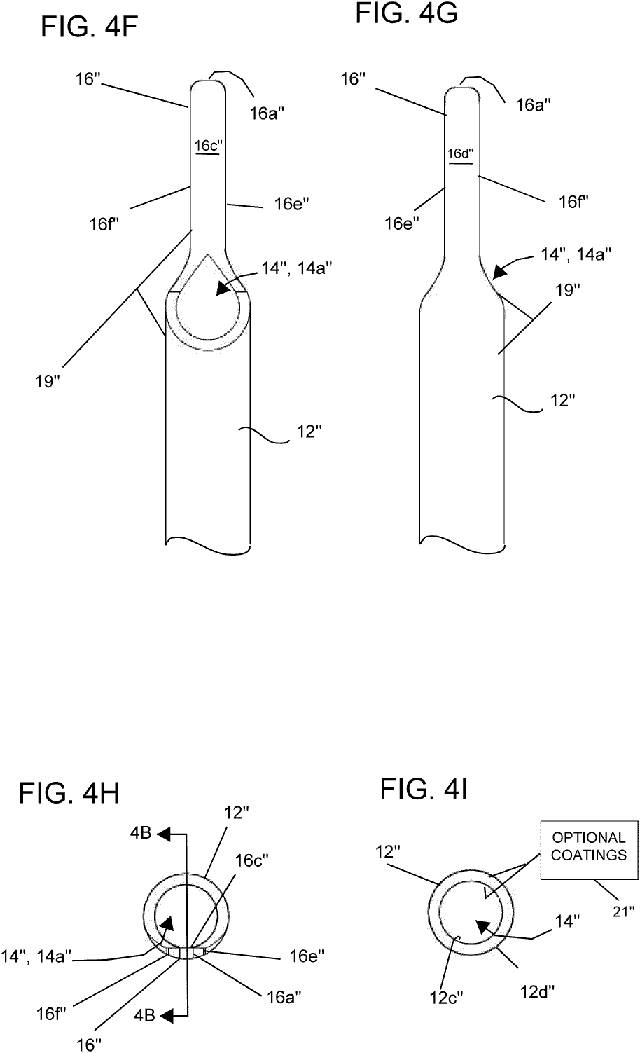

In the embodiment of FIGS. 2A and 2B, note that the end wall 12b of the tubular body 12 lies in a generally vertical plane (FIG. 2D) and is generally perpendicular to the cautery tip 16. In this regard, the angle θ (FIG. 2D) is about 90°, but it could lie in a different angle, such as is illustrated in FIGS. 4A-4E.

In the illustration being described, it should be understood that the switches 20a and 20b are conventional and as mentioned earlier, may be on different AC or DC frequencies, and are illustrated in the electrocautery instrument and system 10 and operatively coupled to the operating electronics and power supply 22, which has its own power level control of the type that is conventionally known. It should also be understood that the electrocautery instrument and system 10 may also comprise an optional foot switch 24 (FIG. 2B) that is operatively coupled to the electrocautery instrument and system 10. During operation, the user sets a generator power, which is not shown, but is part of the operating electronics and power supply 22, to a desired cauterization or power level that will achieve the desired surgical effect. In general, it is preferable to use the lowest possible generator setting which is conventionally known. Once the power level or generator level setting is set, the surgeon actuates switch 20a to turn the electrocautery instrument and system 10 on. The surgeon depresses the activation switch 20a which causes the current to flow through the conductive tubular body 12 and cautery tip 16 to perform the desired cauterization. Again, the activation of the electrocautery instrument and system 10 can be through the switch 20a or through the foot switch 24 in a manner that is conventionally known. It should be understood that a constant current may be applied to the cautery tip 16 and/or an intermittent current may be applied to the cautery tip 16 during the cautery process. As is conventionally known, a continuous current or power to the cautery tip 16 may be useful when cutting tissue, whereas an intermittent current or power applied to the cautery tip 16 is effective for cauterizing tissue of the patient.

It is important to understand that during the cautery process, vapor and smoke are generated. Note, however, that the generally centralized passageway or lumen 14 and inlet 14a are situated in close proximity to the cautery extension 19 and the cautery tip 16. During the cautery process, the vacuum source 18 is energized or activated in a manner conventionally known and creates a negative pressure at area PA1 and at the cautery tip 16 and through the passageway or lumen 14. This causes the smoke and vapors to be evacuated directly into and through the electrocautery instrument and system 10 and tubular body 12. The smoke and vapors are then transmitted to and received by the vacuum source 18. As mentioned earlier, the smoke and vapor may be passed to the decontamination and/or filtration device 20 so that it can be decontaminated, filtered or exhausted (for example, outside the surgical area or into a duct system (not shown)).

Advantageously, the tubular body 12 not only provides a conductive pathway through the electrocautery instrument and system 10 and to the cautery tip 16, but it also defines the passageway or lumen 14 that is generally centralized in the electrocautery instrument and system 10, as illustrated in FIG. 2B. This permits the smoke or vapors generated by the cautery tip 16 to be evacuated directly through the electrode or tubular body 12 itself, which comprises the cautery extension 19 and is formed as part of the tubular body 12 or is coupled thereto. In this regard, the cautery extension 19 is integrally or monolithically formed as part of the tubular body 12 in this embodiment. Alternatively, it is also contemplated that rather than using the monolithically or integrally formed part of the tubular body 12, the cautery extension 19 could be conductively coupled to the tubular body 12 by conventional means, such as by a weld, a snap-fit, a bayonet connection, a press-fit connection, a threaded connection, a conductive adhesive or the like. Moreover, it is also contemplated that the cautery extension 19 could be adapted to be a separate conductive component that is detachably coupled to the tubular body 12. For example, it is also contemplated that the cautery extension 19 could be either a female or a male connector that mates with a male or female connector, respectively, on the tubular body 12. Alternatively, and as described herein, the cautery extension 19 may be integrally or monolithically formed as shown.

The cautery tip 16 comprises an end tip 16a and an elongated blade portion 16b comprising surfaces 16e and 16f, an upper surface 16c (FIGS. 2C-2I), a lower surface 16d coupled or joined by the surfaces 16e and 16f. It should be understood that in general, the surgeon uses the cautery tip 16 and lower surface 16d during the cauterization process and guides the elongated blade portion 16b of the cautery tip 16 and lower surface 16d to the tissue upon which cauterization is desired to be performed. It should be understood, however, that the entire tubular body 12, the cautery extension 19 and the cautery tip 16 are conductive, as mentioned earlier, and the cautery tip 16 and all surfaces 16a-16f may be used during the cautery procedure.

It should be appreciated that the tubular body 12 is manufactured or machined to define or provide the cautery tip 16. FIG. 2C illustrates the removed material in phantom, which leaves the cautery tip 16. Note that the surfaces 16a-16f are manufactured or machined. Surface 16d is generally parallel and the upper surface 16c is generally flat or planar (FIGS. 2F-2I). The lower surface 16d is generally arcuate and has the same radius of curvature as the outer wall 12c of the tubular body 12. However, as will be apparent with the other embodiments described herein, the surfaces 16c, 16d and 16e, 16f do not have to be parallel and one or both of them can be non-parallel, follow a curvature of the tubular body 12, or even comprise a different shape or curvature than the outer wall 12d. Thus, it is contemplated that a single conductive tubular body 12 could be provided and then machined to provide the cautery extension 19 and the cautery tip 16. In this embodiment, the tubular body 12 is defined by the thickness T of at least a portion (such as portion T3 in FIG. 2C) of the inner wall 12c of the tubular body 12, and the surface 16d has a radius that is generally the same as the radius of the tubular body 12. However, if it is desired to provide a particular configuration, the cautery extension 19 and the cautery tip 16 may be manufactured or machined to provide a predetermined or desired shape. Various different shapes are illustrated in the embodiments described later herein relative to FIGS. 10A-10D.

In the illustration being described, the electrocautery instrument and system 10 applies a high frequency alternating current that is applied via either a conventional unipolar or bipolar method. The application of the high frequency alternating current can be applied in a continuous waveform to cut or as an intermittent waveform to coagulate. In one embodiment, cauterization is applied and the circuit’s exit point is a large surface area or conductive pad CP (FIG. 10A) that is typically placed under the buttocks of the patient. In this embodiment, the operating electronics and power supply 22 may utilize a unipolar method in which the tubular body 12 and the integral cautery tip 16 are operatively coupled via the patient to the conductive pad CP as is conventionally known to cause the electrical current to be focused on the cautery tip 16 and to prevent electrical burns to the patient. The amount of heat generated depends on the size of the contact area, power setting and/or frequency of current waveform applied during the surgical procedure. Generally, a constant waveform generates more heat than an intermittent waveform, with the intermittent form being preferred for coagulation, while the constant waveform is preferred for cutting as mentioned earlier.

As is conventionally known, during the monopolar electrocautery process, the current passes through the patient who completes the circuit from the active cautery tip 16 to the patient return electrode or conductive pad CP. In some instances, electrical rhythms of the patient may be a concern or if the patient has, for example, metallic implants.

The electrocautery instrument and system 10 could be adapted for a bipolar electrocautery method (FIG. 10B) which utilizes an alternating current (AC) or a direct current (DC). In this embodiment, the electrocautery instrument and system 10 comprises two distinct conductive, but separate cautery tips 16. The surgical site is placed between the cautery tips 16 and cauterization is performed in a manner conventionally known. FIG. 10B shows a schematic of this embodiment. The two working cautery tips 16 are conductively separated and are movable to cauterize the surgical area of a patient by situating the surgical site between the cautery tips 16 which completes the circuit as is known. Advantageously, this embodiment limits a patient’s exposure to current passing through the body in comparison to the unipolar method described earlier. In this regard, the cautery tips 16 cooperate to perform bipolar cauterization. For a bipolar setup, the current electrode would be split down the center of the cautery extension 19 and the tubular body 12, centrally along the axis of the electrocautery instrument and system 10 with positive and negative connections on each. During this bipolar method, the current passes between the cautery tips 16 (FIG. 10B). In this embodiment, the region of the patient to be cauterized is situated between cautery tips 16 during cutting or cauterization. With the application of a current and waveform, the electrocautery instrument and system 10 cauterizes the area. One advantage of this particular approach is that the current passes through a very small area of the patient and coagulates or cuts the tissue. The bipolar method can be advantageous because it does not disturb the electrical body rhythms of the patient. For example, it does not disturb the electrical body rhythms of the patient’s heart because the current is focused to the area between the cautery tips 16. The bipolar electrocautery method utilizes the cautery tips 16 and electrode whereas the monopolar method utilizes the dispersive and conductive pad CP (FIG. 10A). Both methods utilize the central passageway or lumen 14.

Regardless of which process, bipolar or nonpolar, it should be appreciated that the operating electronics and power supply 22 of the electrocautery instrument and system 10 provide the necessary current and waveforms to either cut or coagulate the tissue of the patient during a surgical procedure.

Advantageously, the electrocautery instrument and system 10 overcomes various disadvantages of the prior art. For example, one disadvantage of prior art systems is obstruction, wherein the vacuum opening comprises a tube that either surrounds the prior art electrocautery instrument or is adjacent to it, which causes visual obstruction of the surgeon’s vision of the prior art electrode tip. Other significant disadvantages that the electrocautery instrument and system 10 facilitates overcoming is that it reduces a distance between the inlet 14a of the passageway or lumen 14 defined by the tubular body 12 from the cautery tip 16. In prior art systems, the smoke plume can escape between its point of generation at the electrode tip and the evacuation opening of the cautery instruments of the prior art.

Yet another issue with the prior art cautery devices is their size. Because the prior art lumens were typically located outside the prior art electrocautery instrument, the evacuating cautery instruments of the prior art were significantly larger and more cumbersome. One advantage of the embodiments being described is that the electrocautery instrument and system 10 is significantly smaller than the prior art devices because the tubular body 12 itself defines the passageway or lumen 14 that runs through the entire electrocautery instrument and system 10 and is generally centrically located in the tubular body 12. The conductive cautery tip 16 with the tubular body 12, also defines the passageway or lumen 14, which is used to evaporate vapor and smoke directly through the tubular body 12. Thus, the tubular body 12 not only is conductive and permits the electrical current from the operating electronics and power supply 22 to pass into the cautery tip 16, but it also enables the integral or monolithic cautery extension 19 to be manufactured from and defined by at least a portion of the tubular body 12. Thus, the electrocautery instrument and system 10 performs multiple functions in addition to a controlled application of electrical current onto a biological tissue of the patient because it also defines the passageway or lumen 14 itself. This greatly reduces the bulk, size and more efficiently evacuates plume and vapors resulting from the cautery process.

As mentioned earlier herein, one problem of the prior art that is overcome by the embodiments being described herein is that the plume of smoke or vapor resulting from the cautery process can escape between its point of generation at the patient. To overcome this issue, the cautery tip 16 and the inlet 14a of the passageway or lumen 14 are proximately relative to each other in order to minimize or eliminate altogether any smoke or vapor from escaping from its point of generation at the cautery tip 16 and the inlet 14a of the passageway or lumen 14.

Notice that the end wall 12b of the tubular body 12 and cautery tip 16 may be adapted to facilitate the collection and evacuation of the smoke or vapor. In this regard, a portion of the tubular body 12 may be adapted to have a predetermined shape that facilitates the collection and evacuation of the smoke or vapor. In this regard, a portion of the end wall 12b, such as a portion 30 illustrated in FIG. 2J, could be a predetermined shape or configuration that facilitates such evacuation of the smoke and vapor. For example, the end wall 12b could be frustoconically or horn-shaped at the portion 30 (FIG. 2J). The portion 30 may also comprise one or more openings, holes or fenestrations (not shown) to also facilitate collection and evacuation of the smoke and vapor.

It should be appreciated, therefore, that the proximity of the end wall 12b and the inlet 14a of the passageway or lumen 14 relative to the cautery extension 19 and the cautery tip 16 enables evacuation of the smoke and vapor directly through the electrocautery instrument and system 10 and the tubular body 12, so the electrocautery instrument and system 10 is highly effective at evacuating the smoke or vapor directly through the electrocautery instrument and system 10 and away from the patient and surgeon to facilitate reducing or eliminating the possible exposure of the surgeon or other personnel from being exposed to the smoke or vapor.

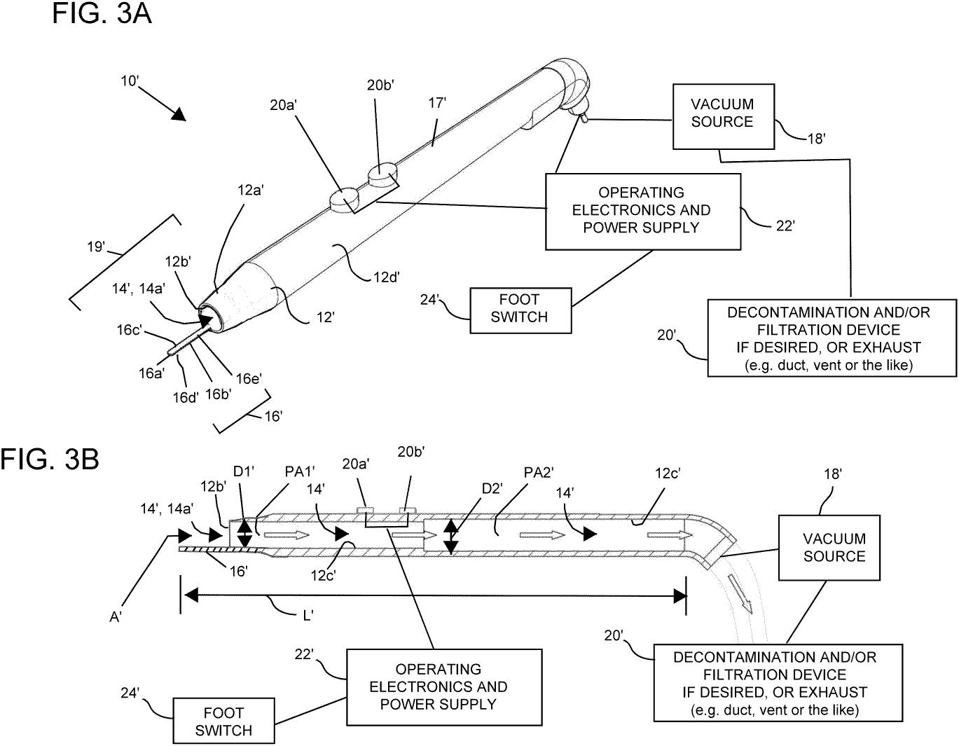

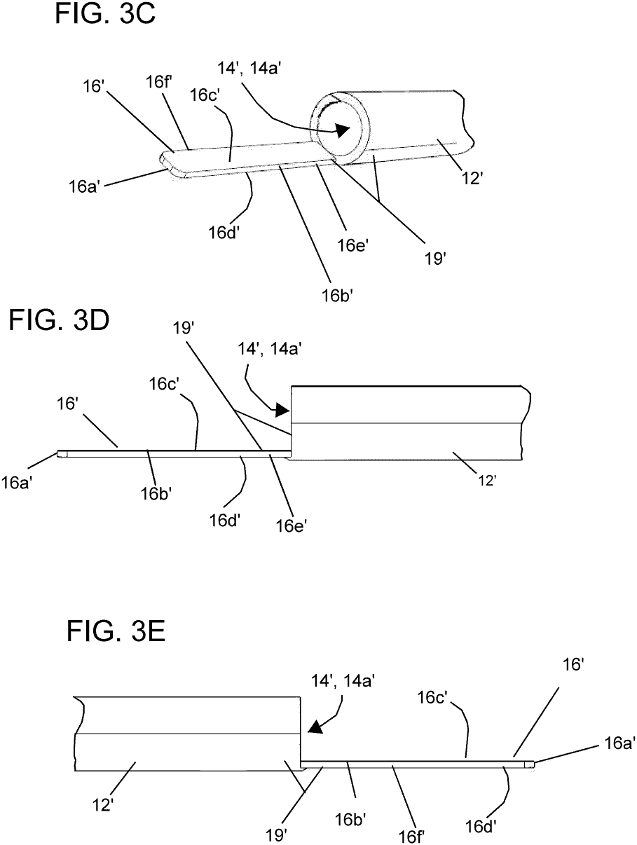

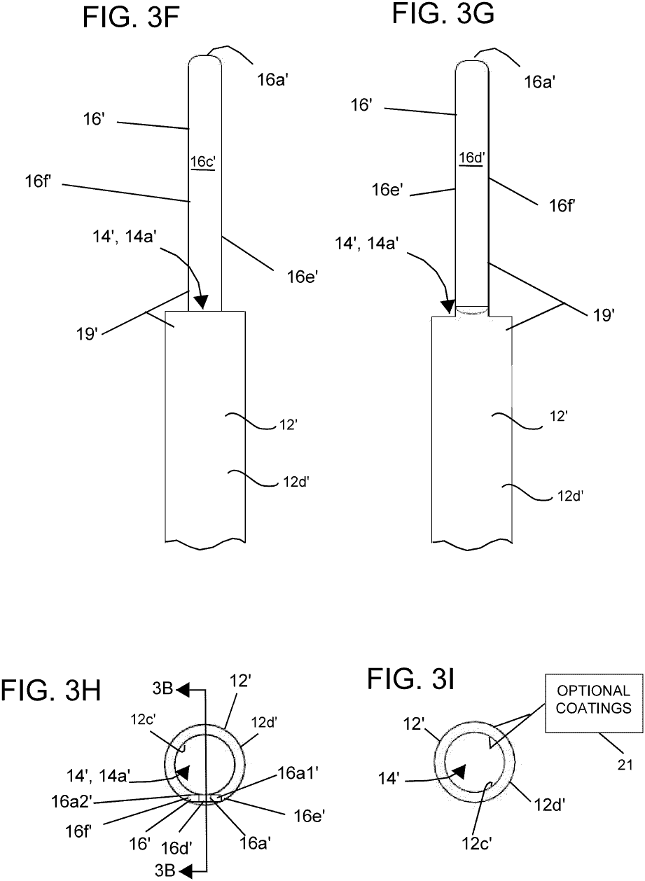

Referring now to FIGS. 3A-10B, other embodiments of the electrocautery instrument and system 10 are shown. In these embodiments, the same or similar parts are identified with the same part numbers, except that one or more prime marks (“ ’ “) have been added to these part numbers for the different embodiment.

In the embodiment of FIGS. 3A-3I, both the upper and lower surfaces 16c′ and 16d′ of the cautery tip 16′ are machined to be generally parallel and generally planar. In contrast, note the embodiment of FIGS. 2A-2J comprises the upper surface 16c that is generally planar, but as mentioned earlier, note that the lower surface 16d is curved or arcuate and follows a circumference or radius of curvature that is generally the same as or similar to the circumference and radius of curvature of the outer wall 12d. Thus, in the embodiment of FIGS. 2A-2J, the lower surface 16d of the cautery tip 16 is not generally planar to the upper surface 16c. Other embodiments show the surface 16d machined as described later herein. The inventor has found that in some applications, it may be preferable to use the flat lower surface 16d′ and the cautery tip 16′ of the embodiment of FIGS. 3A-3I, whereas in other applications or surgeries, it may be preferable to use the embodiment of FIGS. 2A-2J, which provides the generally curved or arcuate lower surface 16d.

FIGS. 3C-3I show perspective and various other views of the embodiment shown in FIGS. 3A-3I. FIGS. 3F and 3G, respectively, are top and bottom views, respectively, of the cautery extension 19′ shown in FIG. 3C. Notice how in the embodiment of FIGS. 2A-2J, the outer wall 12d of the tubular body 12 and a surface 16d1 generally follow the same radius of curvature and circumference and lie in the same cylindrical plane as the lower surface 16d. In contrast, the lower surface 16d′ (FIG. 3H) in the embodiment of FIGS. 3A-3I is generally planar.

The cautery extension 19′ and the cautery tip 16′ may also have lateral portions 16a1′ and 16a2′ (FIG. 3H) that are truncated to reduce or eliminate sharp edges and/or to bring the cautery tip 16′ to a relative point.

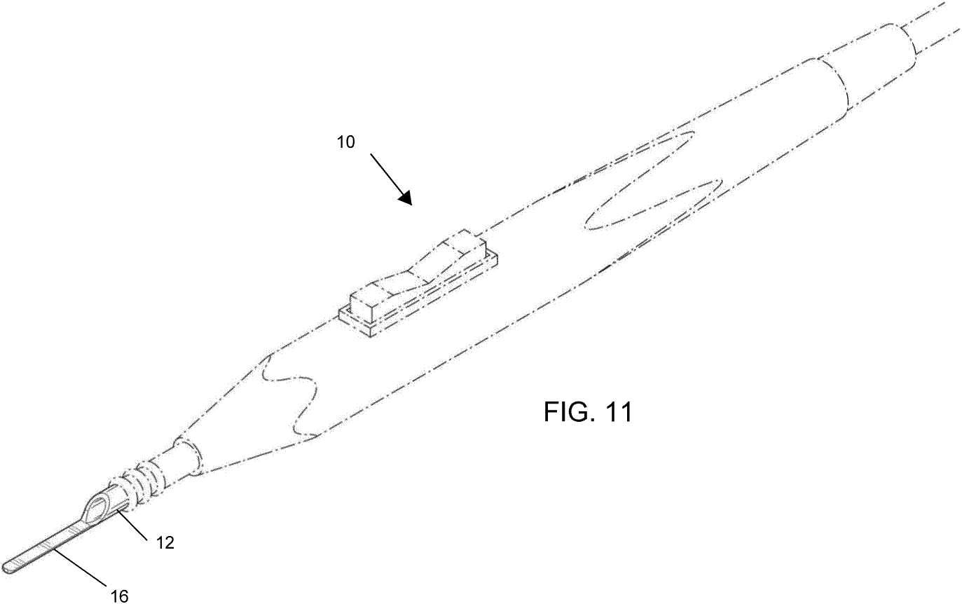

Referring now to FIGS. 4A-4I, another illustrative embodiment is shown. In the embodiment of FIGS. 4A-4I, the same or similar parts are identified with the same part numbers, except that two prime marks (“ ” ”) have been added to these part numbers. FIGS. 11-27 are further details of the cautery tip 16″ shown in FIGS. 4A-4I. The ornamental designs are shown illustrating that the cautery tips 16″ could be any desired dimension and length, depending on the application. The elongated portion of each cautery tip 16″ may define the passageway or lumen 14″ through the electrocautery instrument and system 10″. The cautery tips 16″ may be elongated to define the passageway or lumen 14″ through the electrocautery instrument and system 10″. Alternatively, the cautery tips 16″ can be adapted to be easily changed out with a press fit, threaded, bayonet or other type of conventional connection.

In this embodiment, note that the end wall 12b″ of the cautery extension 19″ is angled and lies at an angle ϕ (FIGS. 4C-4E) that is obtuse, as best illustrated in FIG. 4D. In the embodiment of FIGS. 4A-4I, the end wall 12b″ lies in a plane that is generally obtuse with respect to the cautery tip 16″. FIG. 4D is a side view showing the end wall 12b″, a fillet 42″ and the curved lower surface 16d″ of the cautery tip 16″, which is similar to the cautery tip 16 shown in FIG. 2A, except the surface 16d″ is generally flat and not curved. Thus, the cautery tip 16″ in the embodiment of FIGS. 4A-4I operates similarly to the cautery tip 16, 16′ in the embodiments of FIGS. 2A-3I. The end wall 12b″ is angled at an angle ϕ relative the cautery tip 16″.

Note that the end wall 12b″ has at least a portion that lies in a plane that is generally normal or perpendicular (FIG. 4D) to the cautery tip 16″. In this embodiment, note that the cautery extension 19″ comprises the tubular body 12″ and cautery tip 16″. In this embodiment however, an area 40′ between the cautery tip 16′ and the cautery extension 19″ is strengthened by a fillet 42″. In this regard, notice that the fillet 42″ rounds off and strengthens the end wall 12b″, the cautery tip 16″ and cautery extension 19″. In contrast, the embodiments of FIGS. 2A-3I do not utilize the fillet 42″. Thus, note that the embodiments of FIGS. 2A-3I were conventionally processed to not comprise the fillet 42″, but rather a roughly 90 degree angle between the end wall 12b″ and the cautery tip 16″.

In this embodiment because the wall 12b″ is angled at the angle ϕ, the general dimension of the inlet end 14a″ of the passageway or lumen 14″ is generally elliptical and slightly larger than the size of the inlets 14a and 14a′ of the prior embodiments described herein. The angled end wall 12b″ facilitates providing a greater clearance in an area 40″ illustrated in FIG. 4D.

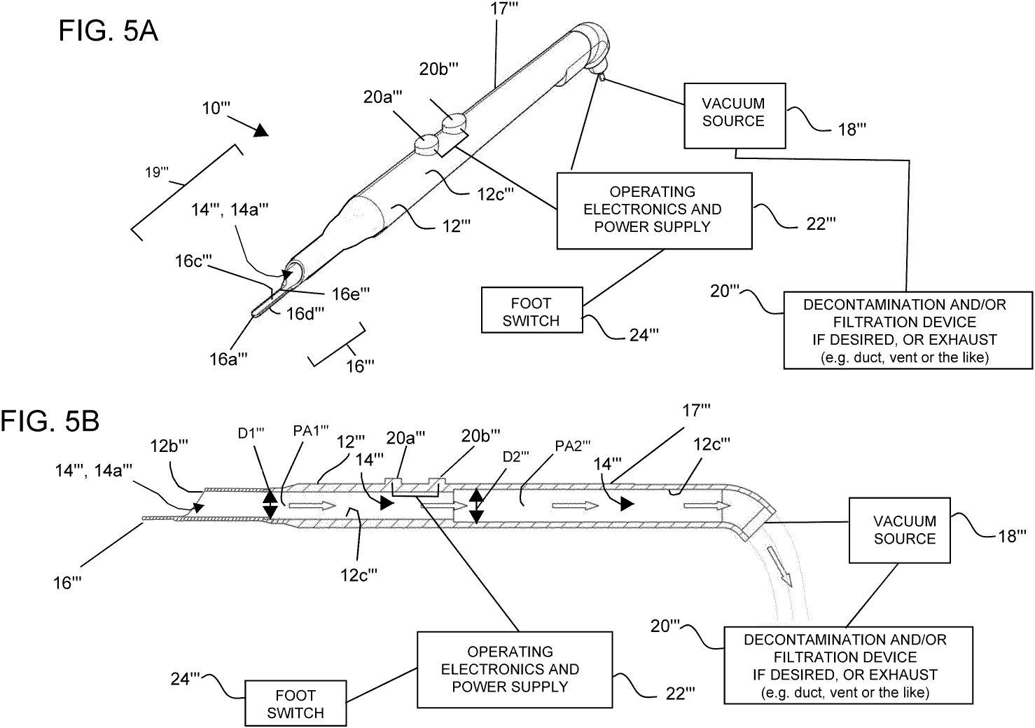

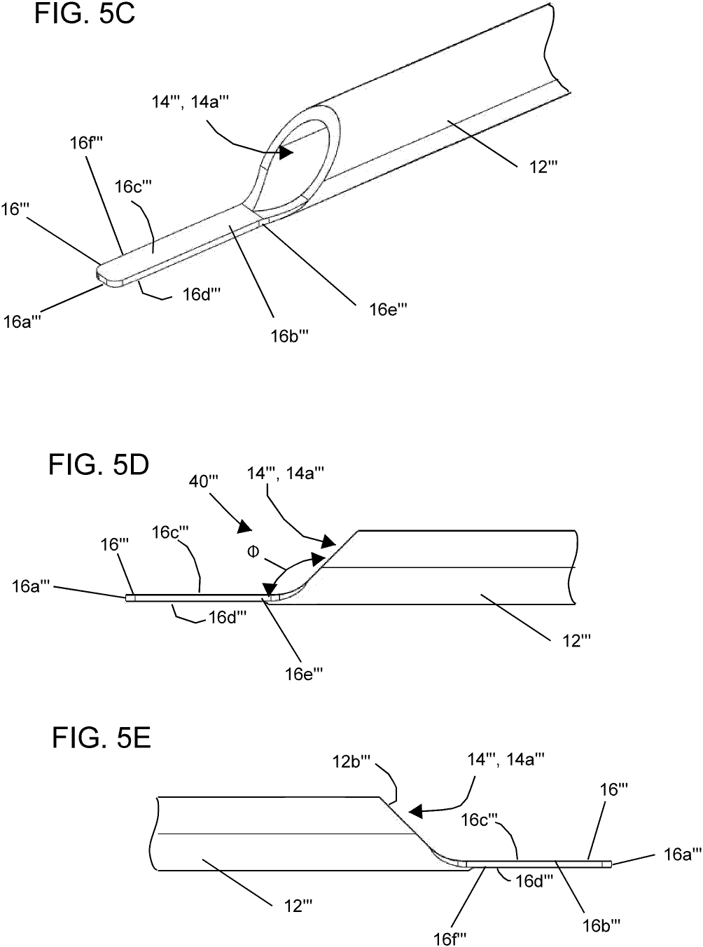

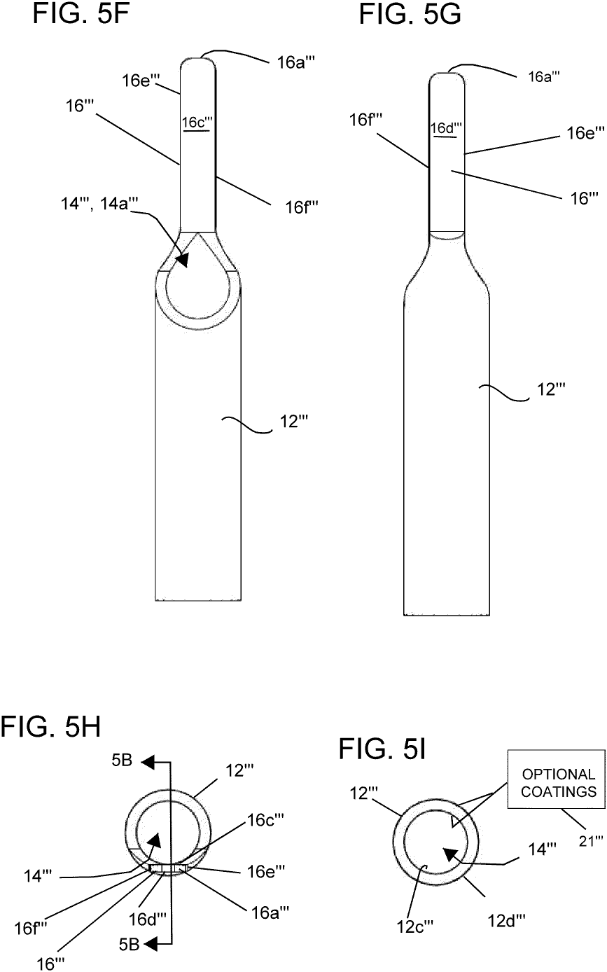

Referring now to FIGS. 5A-5I, still another embodiment of the electrocautery instrument and system 10‴ is shown. In the embodiment of FIGS. 5A-5I, the same or similar parts are identified with the same part numbers, except that three prime marks (“ ’’’ ”) have been added to these part numbers for ease of illustration.

This embodiment is similar to the embodiment of FIGS. 4A-4I, except that the bottom surface 16d‴ has been machined to be generally planar and generally parallel to the upper surface 16c‴ like the embodiment of FIGS. 3A-3I. As with the embodiment of FIGS. 4A-4I, note that the end wall 12b‴ is angled at an obtuse angle Φ (FIG. 5D) relative to the upper surface 16c‴ to again provide the clearance area 40‴ for the convenience of the surgeon or user. As with the prior embodiment of FIGS. 3A-3I, the cautery tip 16‴ of the cautery extension 19‴ could also be machined to be relieved of any sharp corners and to be rounded as illustrated in FIGS. 5F and 5G, for example. As with prior embodiments, all surfaces 16a‴-16f‴ of the cautery tip 16‴ may be used for cauterizing.

Advantageously, the cautery tip 16‴ of the cautery extension 19‴ is adapted to provide means and apparatus for cauterizing a desired location or area on the patient. In the embodiment described in FIGS. 5A-5I, the cautery tip 16‴ is generally linear and substantially co-linear with the end wall 12b‴ of the tubular body 12‴. In the prior embodiments, such as the embodiment of FIGS. 2A-2J, the cautery extension 16 was generally elongated and generally linear and co-linear with the end wall 12b of the tubular body 12 and integral with or monolithically formed with the end wall 12b as described earlier herein. As mentioned earlier, in order to provide more operative clearance at the area 40‴, the end wall 12b‴ may be angled at the angle Φ as described earlier herein.

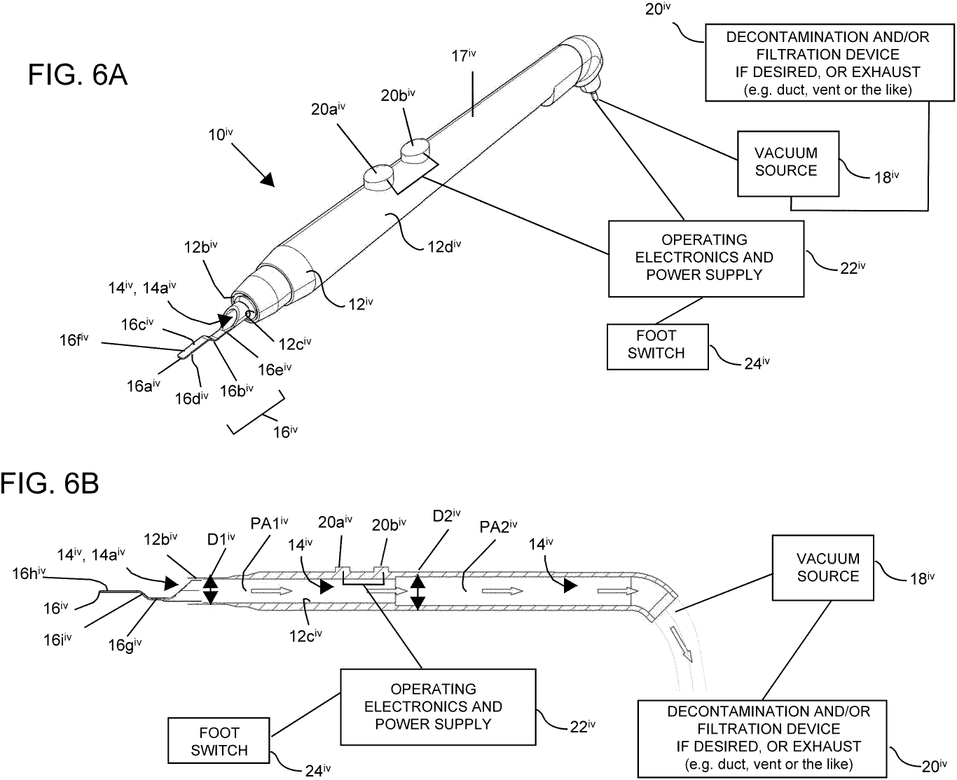

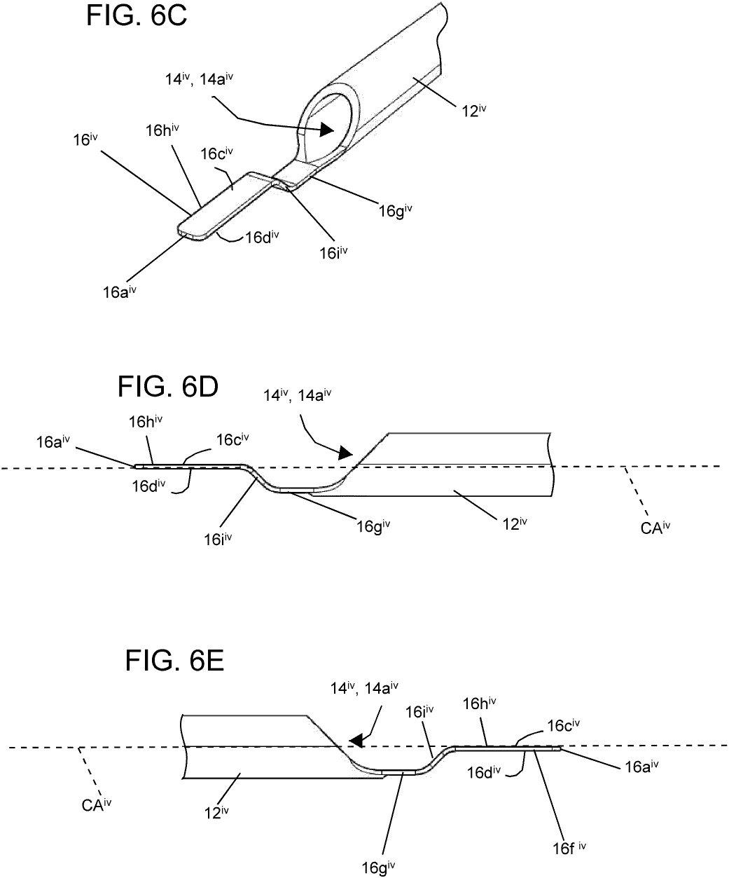

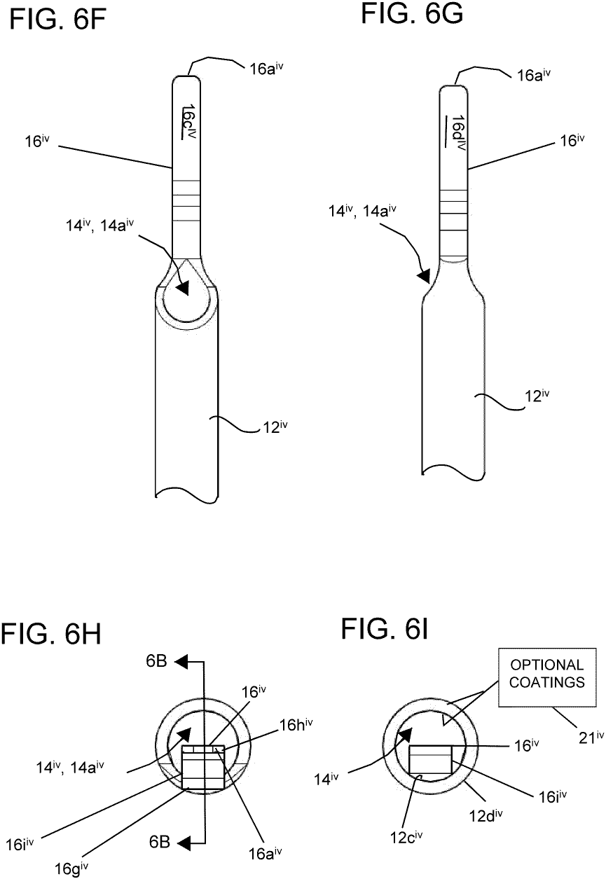

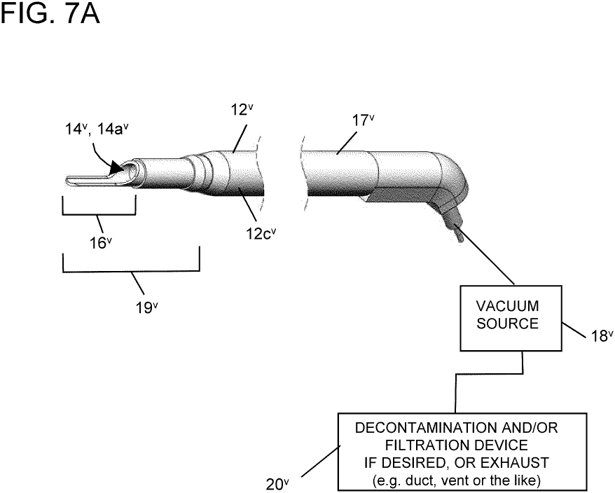

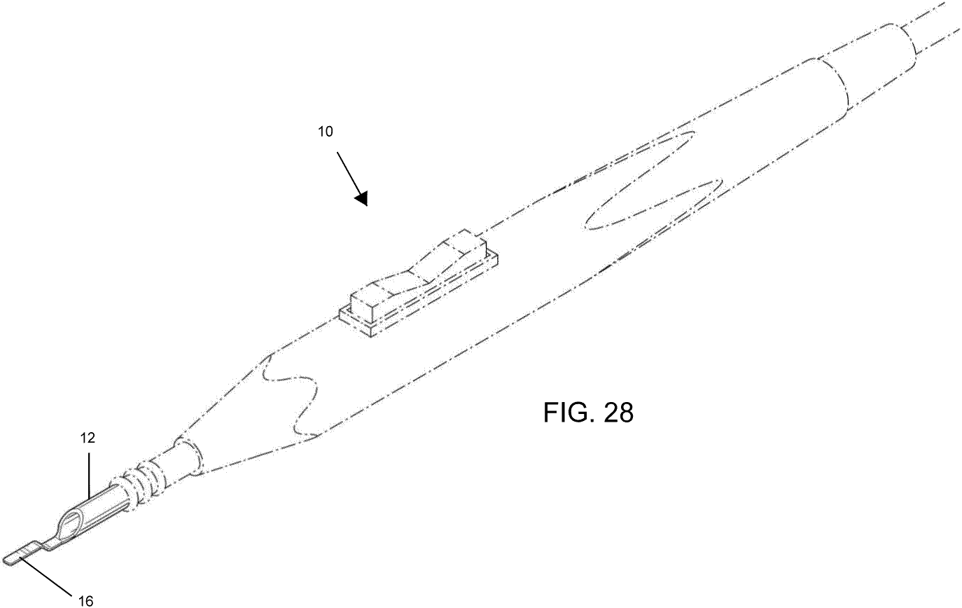

The inventor also contemplates that the cautery tip 16‴ or other portion of the cautery extension 19‴ may be non-linear, stepped, angled, curved or an arcuate shape in multiple planes. The embodiment of FIGS. 6A-6I illustrates this feature. In the embodiment of FIGS. 6A-6I, the same or similar parts are identified with the same part numbers, except that a prime mark iv (^{“} ^{iv} ^{“}) has been added to these part numbers for ease of illustration. FIGS. 28-44 are further details of the cautery tip 16^{iv} shown in FIGS. 6A-6I. The ornamental designs are shown illustrating that the cautery tips 16^{iv} could be any desired dimension and length, depending on the application. The elongated portion of each cautery tip 16^{iv} may define the passageway or lumen 14^{iv} through the electrocautery instrument and system 10^{iv}. The cautery tips 16^{iv} may be elongated to define the passageway or lumen 14^{iv} through the electrocautery instrument and system 10^{iv}. Alternatively, the cautery tips 16^{iv} can be adapted to be easily changed out with a press fit, threaded, bayonet or other type of conventional connection.

In FIG. 6A, the cautery tip 16^{iv} is stepped along its axis. Note that the cautery tip 16^{iv} in this embodiment comprises the upper surface 16c^{iv} which is integrally coupled to or monolithically or integrally formed in the end wall 12b^{iv}. The cautery tip 16^{iv} further comprises a second portion 16h^{iv} (FIGS. 6C-6E) and a joining portion 16i^{iv} that joins the portions 16g^{iv} and 16h^{iv}. In the illustration being described, the portion 16i^{iv} may be a vertical step or angled as illustrated in FIGS. 6C-6E.

Note that with this embodiment, the portion 16h^{iv} of the cautery tip 16^{iv} lies in a plane that is generally co-axial with a center axis CA (FIGS. 6D-6E) of the electrocautery instrument and system 10^{iv}. The consequence of this is that the portion 16h^{iv} is generally centrally located with the passageway or lumen 14^{iv} and in proximity to the end wall 12b^{iv} and the inlet 14a^{iv} of the passageway or lumen 14^{iv} at the end of the electrocautery instrument and system 10^{iv} so that when cauterization occurs, any smoke or vapor generated during the cautery process around cautery tip 16^{iv} is collected into the inlet 14a^{iv} and passageway or lumen 14^{iv}.

The embodiment of FIGS. 6A-6I is adapted to provide means and apparatus for causing the vacuum or evacuation with negative pressure and suction that generally equally surrounds the cautery tip 16^{iv}. In contrast, note that in the embodiments of FIGS. 2A-5I, a substantial amount of the negative pressure at the cautery tip 16^{iv} is above the surfaces (e.g., above surface 16c, 16c′, 16c″, 16c‴ and the like). In contrast, the embodiment of FIG. 6A provides a more even distribution of the negative pressure or vacuum around the entire cautery tip 16^{iv} so that the negative pressure surrounding the portion surface 16c^{iv} and 16e^{iv} is substantially constant or the same. One advantage of the centrally positioned cautery tip 16^{iv} along the central axis of the electrocautery instrument and system 10 shown in FIG. 6A, is that it potentially provides better manual control by the surgeon. The coagulation zone will remain central, even if the electrocautery instrument and system 10 is rotated in the surgeons’ hand or the angle of surgical approach differs. For the offset electrode, rotation of the electrocautery instrument and system 10 will move the coagulation zone which may not be desirable. An analogy would be how an off-axis tip on a ballpoint pen would make writing difficult. As with prior embodiments, the upper surface 16c^{iv} and lower surface 16d^{iv} may be generally curved, arcuate or planar as illustrated in FIGS. 6C-6E so that the lower surface 16d^{iv} is generally planar similar to the surfaces 16d, 16d′, 16d″ and 16d‴ of prior embodiments.

As best illustrated in FIGS. 6G and 6H, note that the working surfaces 16c^{iv} and 16d^{iv} are generally centrally located and generally co-axial with the axis CA of the passageway or lumen 14^{iv}. This embodiment is advantageous in that it generally centrally aligns the working surfaces 16c^{iv} and 16d^{iv} relative to the inlet 14a^{iv} of the passageway or lumen 14^{IV} to more efficiently evacuate smoke and vapor created by the cautery process.

As with the embodiment described earlier herein relative to FIGS. 2A-2J, the embodiments of FIGS. 3A-6I operate similarly. During use, the surgeon or operator depresses the actuation button 20a^{iv} to cause the current to pass through the conductive tubular body 12^{iv} and through the conductive cautery tip 16^{iv}. When applied to the patient, the patient completes the circuit and the current returns through the conductive pad CP (FIGS. 10A and 10B).

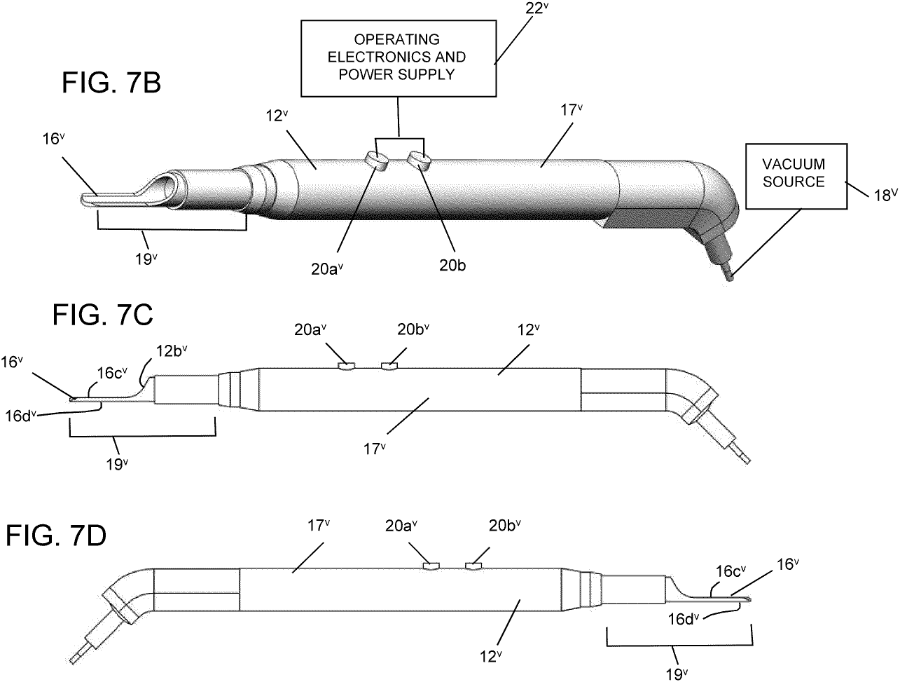

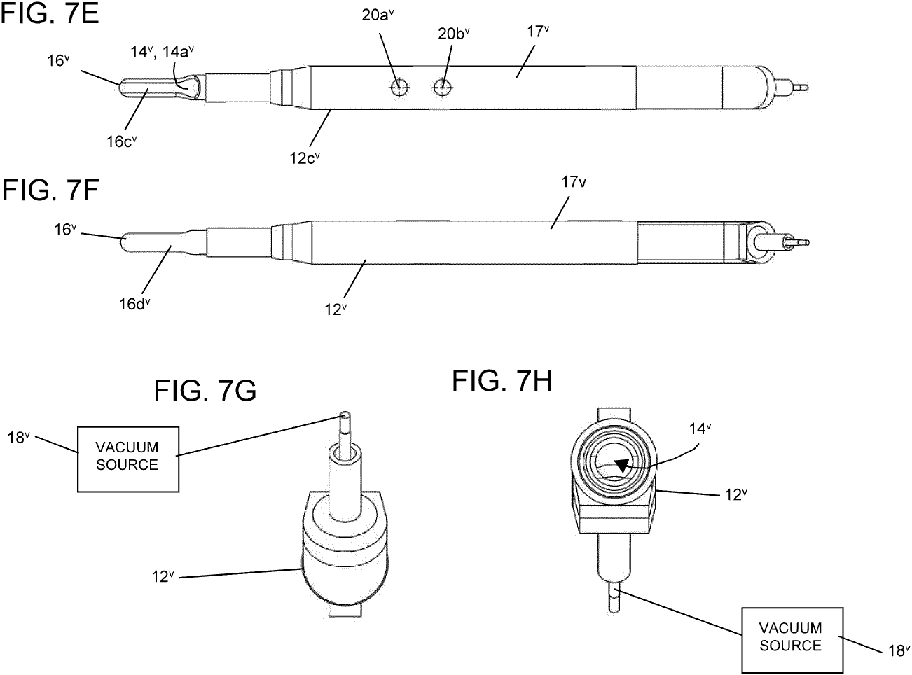

Referring now to FIGS. 7A-7H, another embodiment is shown. This embodiment is similar to the embodiment shown in FIGS. 4A-4I.In the embodiment of FIGS. 7A-7H, the same or similar parts are identified with the same part numbers, except that prime mark v (^{“ v “}) has been added to these part numbers for ease of illustration.

As with other embodiments, note that the cautery extension 19^{v} is monolithically formed or integral with the end wall 12b^{v} as illustrated. In this embodiment, note that neither the upper surface 16c^{v} nor the lower surface 16d^{v} are machined to be generally planar as with the embodiment shown in FIGS. 2A-4I. Note also that the end wall 12b^{v} is angled or tilted to the angle ϕ as described earlier herein relative to the embodiments of FIGS. 4A-4I.Advantageously, this embodiment provides a curved or arcuate upper surface 16c^{v} and a curved or arcuate lower surface 16d^{v}. The surfaces 16c^{v} and 16d^{v} comprise a radius of curvature that is the same or generally similar to the radius of curvature of the inner wall 12c^{v} and outer wall 12d^{v}, respectively, of the tubular body 12^{v}. Advantageously, as with the embodiment of FIGS. 4A-4I, the tubular end wall portion 12a^{v} of the tubular body 12^{v} is machined to provide the angled end wall 12b^{v} that lies at the predetermined angle ϕ relative to the cautery tip 16^{v}.

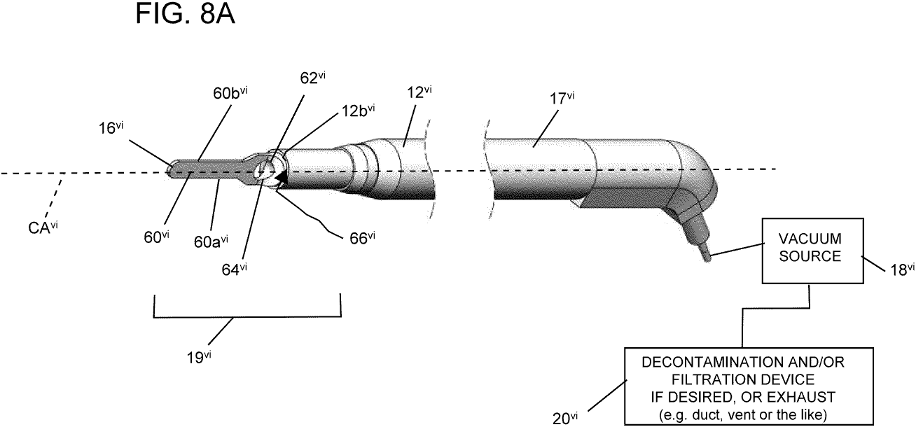

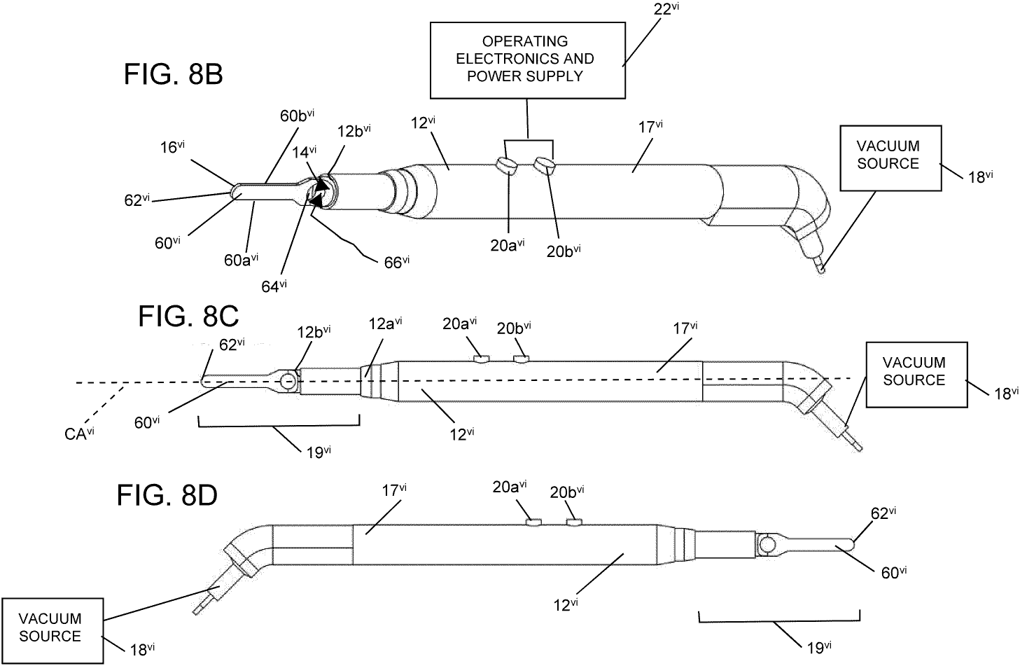

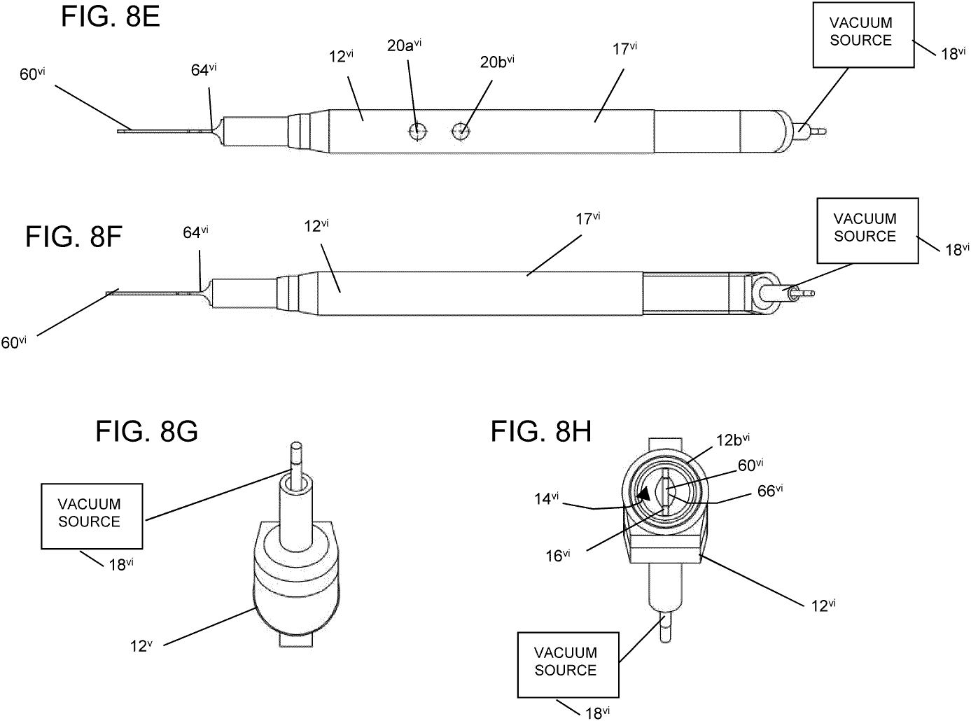

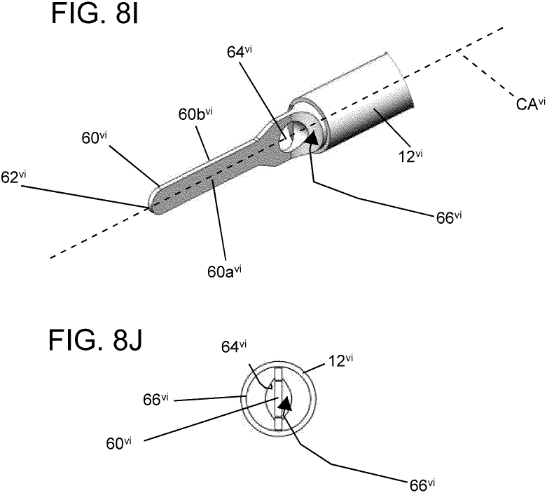

Referring now to FIGS. 8A-8J, another embodiment is shown. In the embodiment of FIGS. 8A-8J, the same or similar parts are identified with the same part numbers, except that prime mark vi (^{“} ^{vi} ^{“}) has been added to these part numbers for ease of illustration.

In this embodiment, the cautery extension 19^{vi} is adapted, machined or formed from the tubular body 12^{vi} and the cautery tip 16^{vi} is generally vertically aligned and defines a central blade 60^{vi}. The blade 60^{vi} is integrally or monolithically formed or it may be provided to be detachably secured to the end wall 12b^{vi}. Thus, the blade 60^{vi} could be integral with, monolithically formed or detachably coupled to the tubular body 12^{vi}. For example, the cautery extension 19^{vi} may comprise a threaded male connection (not shown) that is received in a female threaded connection (not shown) or it may be press-fit into the tubular end wall portion 12a^{vi} (FIG. 8C) of the tubular body 12^{vi} to complete a conductive connection. It is important to note that the cautery extension 19^{vi} is conductive and a conductive connection exists or occurs between the cautery extension 19^{vi} and the tubular body 12^{vi}. In this illustration, the cautery extension 19^{vi} defines or comprises the blade 60^{vi} which is generally vertical and has a first surface or side 60a^{vi} and a generally opposing second surface or side 60b^{vi}, both of which are generally planar and parallel. Note that the blade 60^{vi} is generally centrally located on axis CA (FIG. 8C) of the passageway or lumen 14^{vi}. In this regard, notice that at least a portion of the blade 60^{vi} may lie co-axially with the axis CA (FIGS. 8C and 8I) of the passageway or lumen 14^{vi} as illustrated. It should be understood, however, that the blade 60^{vi} could be offset from the axis CA, such is illustrated in one or more of the prior embodiments described herein. Note that the blade 60^{vi} is conductive and comprises a generally cylindrical end 62^{vi} that is integral with or monolithically formed with or coupled to the conductive tubular body 12^{vi} so that current flows through the tubular body 12^{vi} and into the cautery tip 16^{vi}.

Notice that to facilitate smoke and vapor evacuation and to create a negative pressure and vacuum around the blade 60^{vi}, the cautery extension 19^{vi}, at least in part, comprises at least one wall 64^{vi} (best illustrated in FIGS. 8I-8J) that defines at least one fenestration or opening 66^{vi}. The cautery extension 19^{vi} comprises the at least one fenestration or opening 66^{vi} that defines the inlet 14a^{vi} into the passageway or lumen 14^{vi}. The passageway or lumen 14^{vi} causes the smoke and vapor to be evacuated into and through the passageway or lumen 14^{vi} and operates similar to the other embodiments described herein. Notice in FIGS. 8E, 8F and 8H-8I, that the blade 60^{vi} is generally centrally located and generally co-axial with the passageway or lumen 14^{vi}, which facilitates evacuating smoke and vapor from the cautery site from either side 60a^{vi} or 60b^{vi} of the blade 60^{vi}.

It should now be appreciated that the cautery extension 19^{vi}, cautery tip 16^{vi} or blade 60^{vi} and the like are preferably integral with or monolithically formed as part of the tubular body 12^{vi}, but they could be detachably coupled to the tubular body 12^{vi}, by conventional means, such as a threaded connection, bayonet connection, press-fit, conductive adhesive or the like, all of which are mentioned earlier herein.

The embodiments shown in FIGS. 2A-8J illustrate various configurations of the cautery extension 19 and cautery tip 16 and that the cautery extension 19 comprises the cautery tip 16 and may be selected from a kit or group of one or more predetermined shapes shown and described earlier herein.

The embodiments described herein show the cautery tips 16, 16′, 16″, 16‴, 16^{iv}, 16^{v} and 16^{vi} are shaped as illustrated in the FIGS. 2A-8I. It should be understood that the cautery tips 16, 16′, 16″, 16‴, 16^{iv}, 16^{v} and 16^{vi} could be a different predetermined shape as selected by the user.

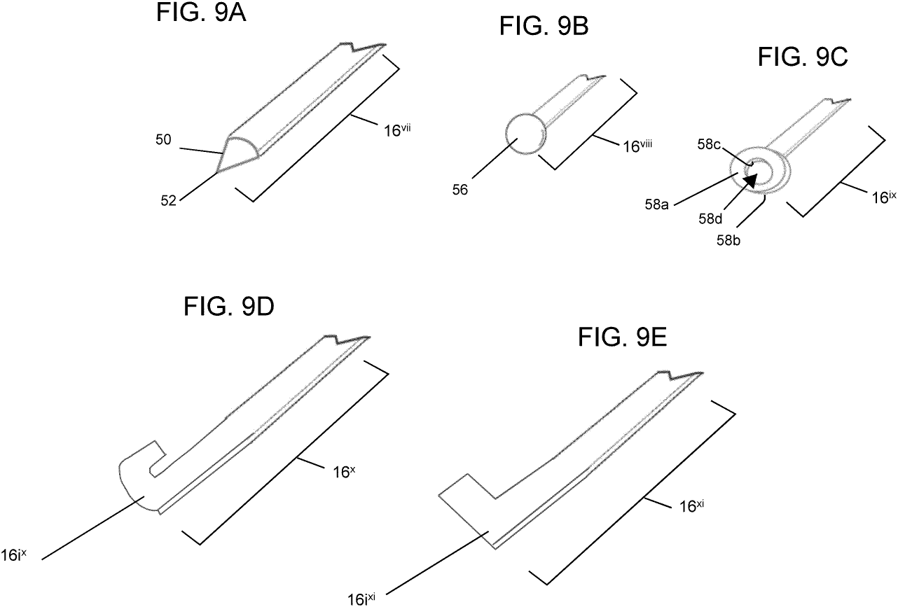

Alternatively, it should be appreciated that the cautery extension 19 and cautery tip 16 may be adapted to comprise other predetermined shapes or configurations as desired. In the embodiments of FIGS. 9A-9E, the same or similar parts are identified with the same part numbers, except that prime marks vii, viii, ix, x or xi (^{“vii,} ^{viii,} ^{ix,} ^{x, xi“}) have been added to these part numbers for ease of illustration. Some representative exemplary predetermined shapes or configurations are illustrated in FIGS. 9A-9E, with it being understood that the cautery tips 16 ^{vii,} ^{viii,} ^{ix,} ^{x,} ^{xi} are part of the cautery extension portions 19 described earlier herein, which are coupled to, integral with or monolithically formed as part of the tubular body 12 as described earlier herein, and are adapted to be conductively connected to, integral with or monolithically formed with the end wall 12b described earlier herein. In FIG. 9A, the cautery tip 16^{vii} may comprise an extension tip 50 having a point 52.

FIG. 9B illustrates another embodiment where the cautery tip 16^{viii} has a ball end 56 as shown. FIG. 9C illustrates the machined cautery tip 16^{ix} having an upper surface 58a and a generally opposed lower surface 58b both of which are planar and generally parallel. This machined cautery extension 16^{ix} comprises a generally circular inner wall 58c that defines an aperture 58d for use during the cautery process.

FIG. 9D illustrates a cautery tip 19^{x} comprising a generally U-shaped or J-shaped elongated portion 16b^{x}. Again, this U-shaped or J-shaped elongated portion 16i^{x} and cautery tip 16^{x} are integrally or monolithically formed with the end wall 12b as described earlier herein. Similarly, FIG. 9E illustrates an L-shaped or hook-shaped elongated portion 16i^{xi}.

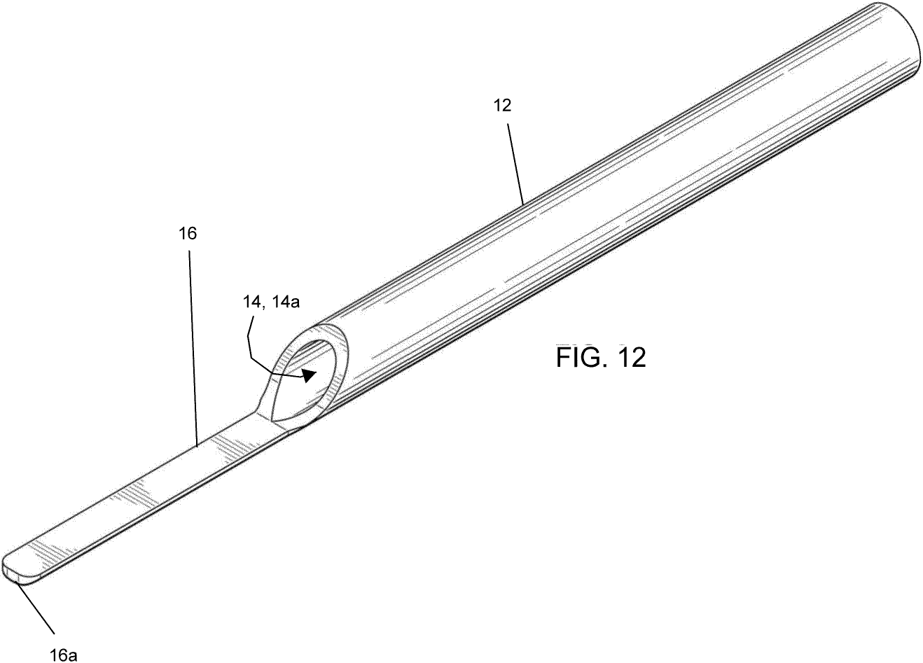

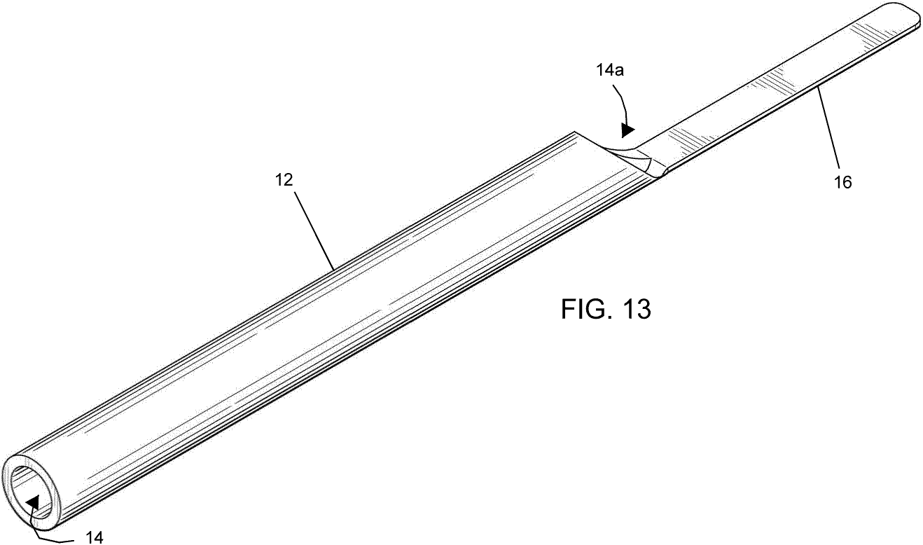

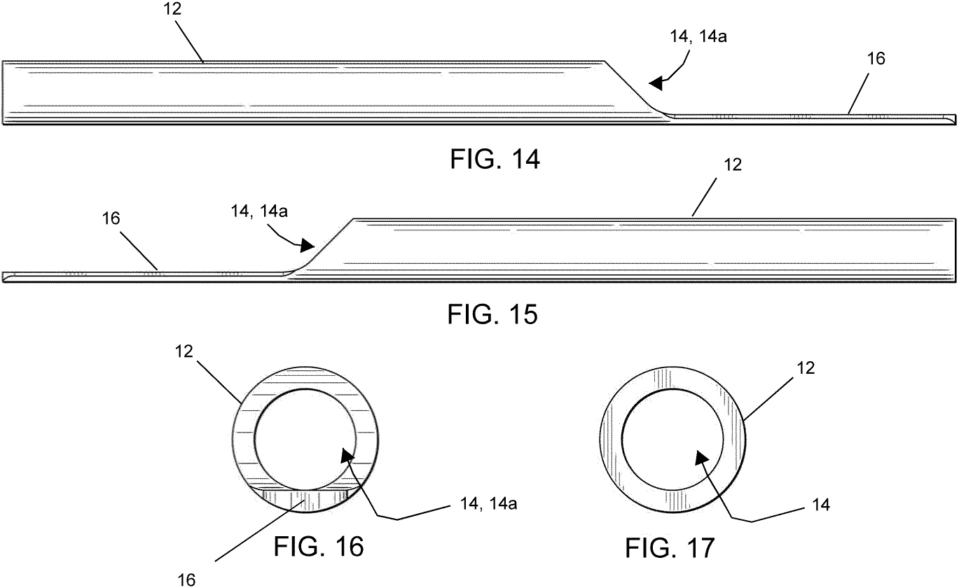

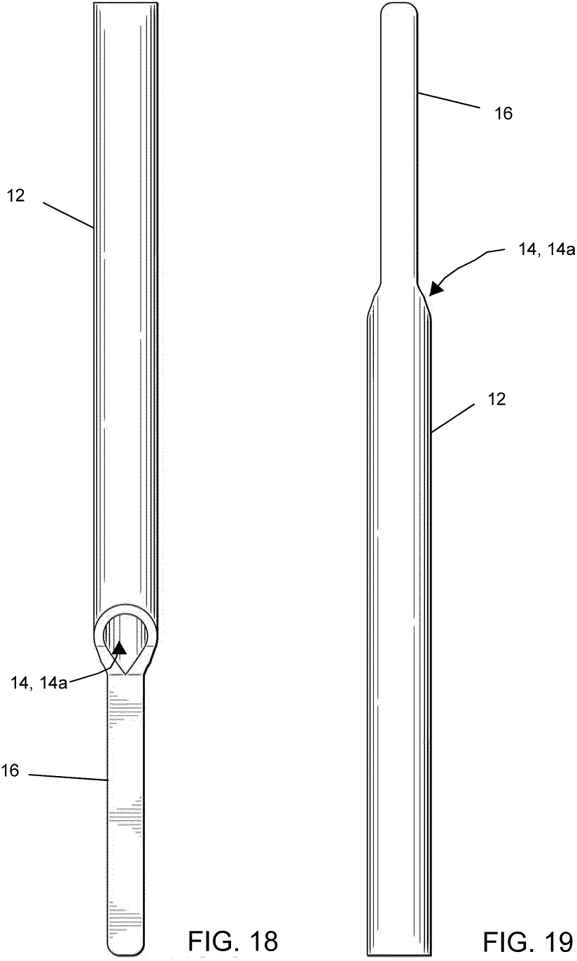

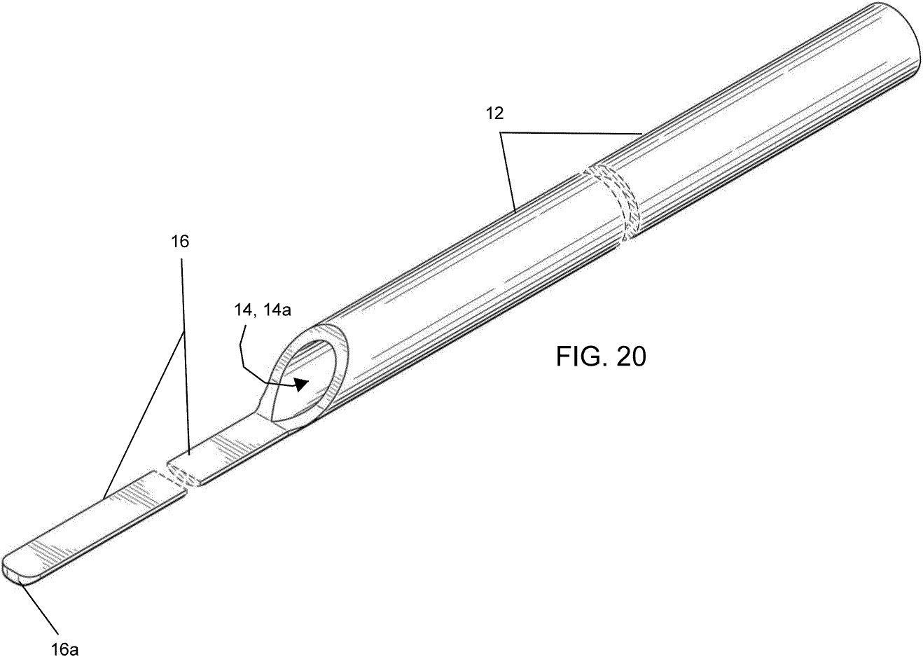

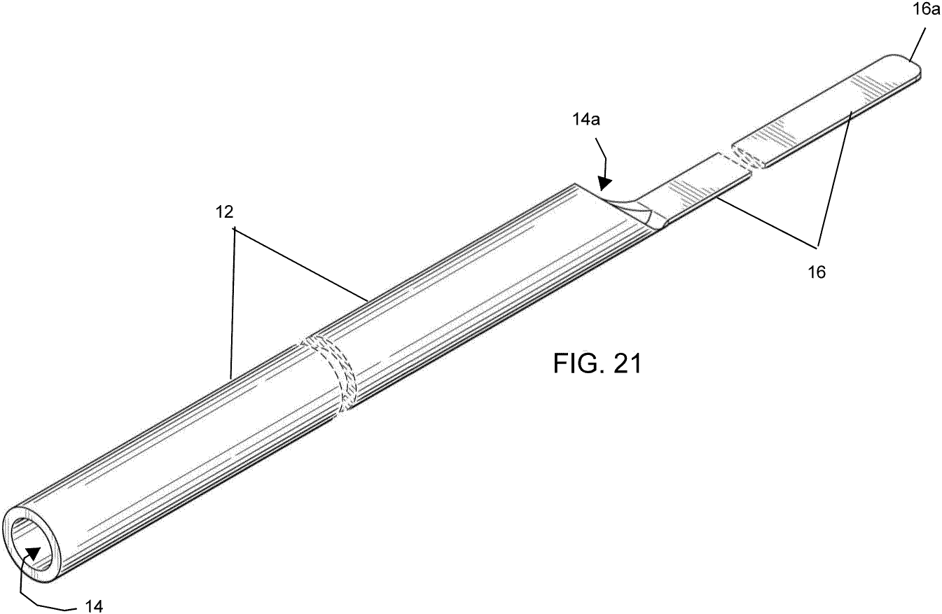

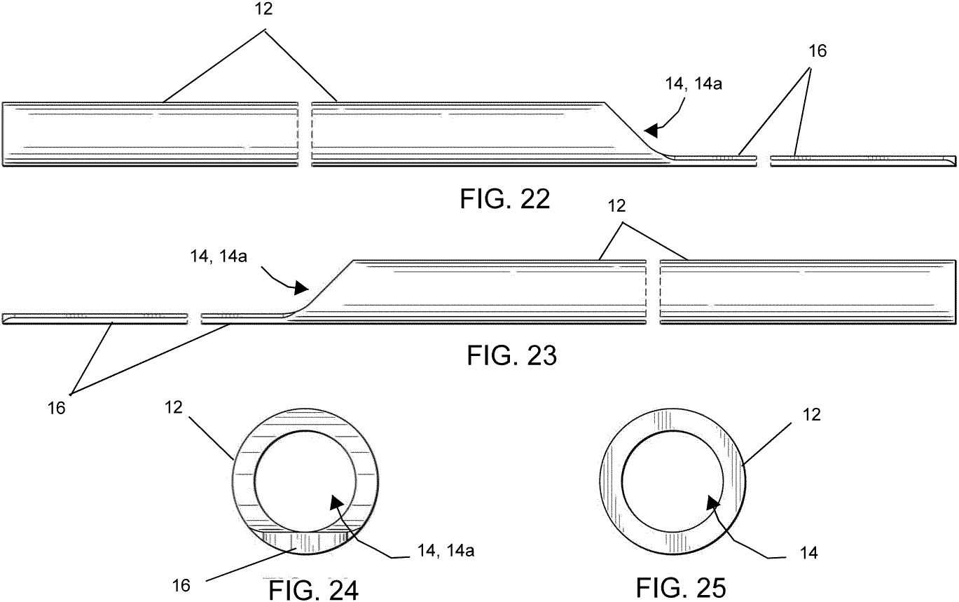

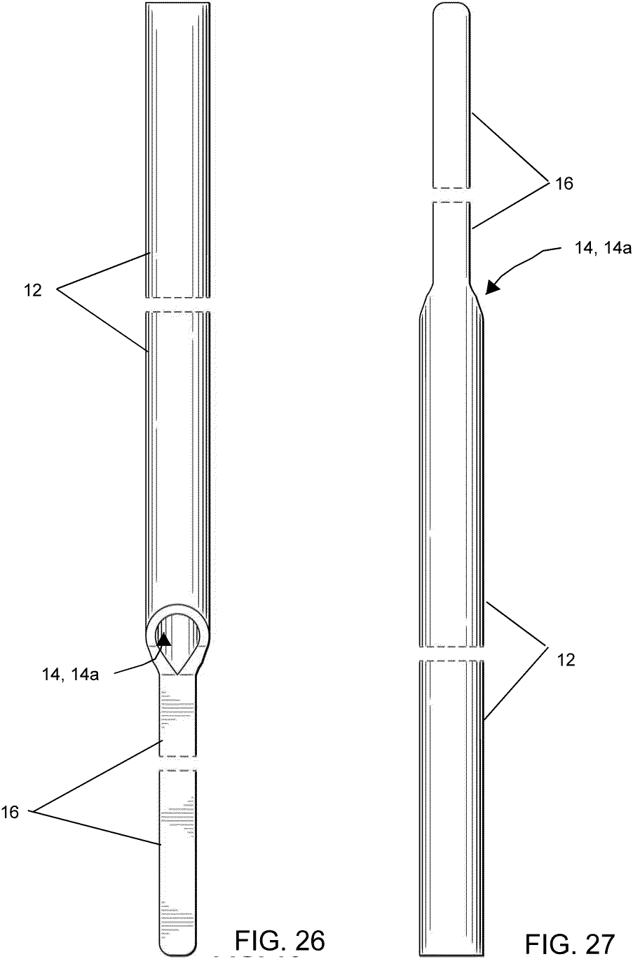

FIGS. 11-27 show the ornamental design of a tapered cautery tip 16 depicted in FIGS. 4A-4I for use with the electrocautery instrument and system 10. FIG. 11 is a perspective view of a tapered cautery tip as it is used with an electrocautery device. FIG. 12 is a right front perspective view of the cautery tip of FIG. 11. FIG. 13 is a left side rear perspective view of the cautery tip of FIG. 11. FIG. 14 is a left side view of the cautery tip of FIG. 11. FIG. 15 is a right side view of the cautery tip of FIG. 11. FIG. 16 is a front view of the cautery tip of FIG. 11. FIG. 17 is a rear view of the cautery tip of FIG. 11. FIG. 18 is a top view of the cautery tip of FIG. 11. FIG. 19 is a bottom view of the cautery tip of FIG. 11. FIG. 20 is a right front perspective view of the cautery tip of FIG. 11 shown with symbolic breaks in its length. FIG. 21 is left rear perspective view of the cautery tip of FIG. 11 shown with symbolic breaks in its length. FIG. 22 is a left side view of the cautery tip of FIG. 11 shown with symbolic breaks in its length. FIG. 23 is a right side view of the cautery tip of FIG. 11 shown with symbolic breaks in its length. FIG. 24 is a front view of the cautery tip of FIG. 11 shown with symbolic breaks in its length. FIG. 25 is a rear view of the cautery tip of FIG. 11 shown with symbolic breaks in its length. FIG. 26 is a top view of the cautery tip of FIG. 11 shown with symbolic breaks in its length. FIG. 27 is a bottom view of the cautery tip of FIG. 11 shown with symbolic breaks in its length.

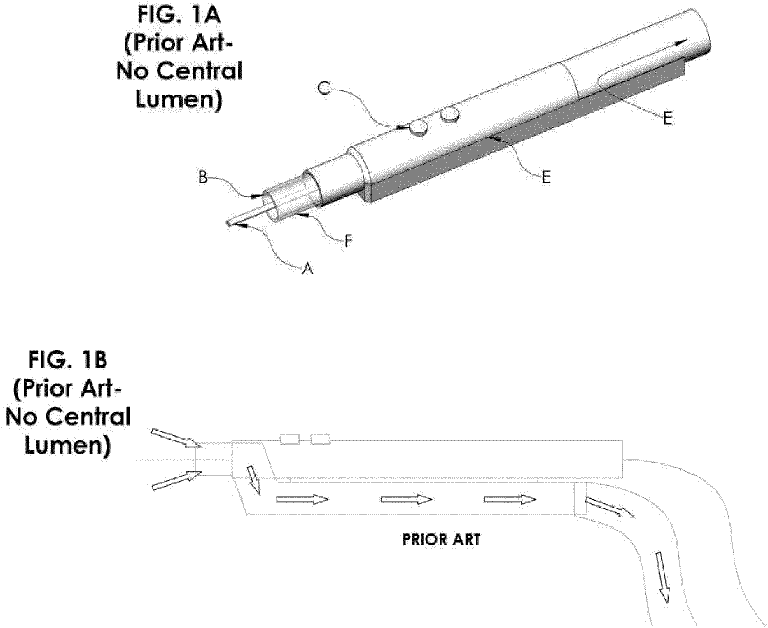

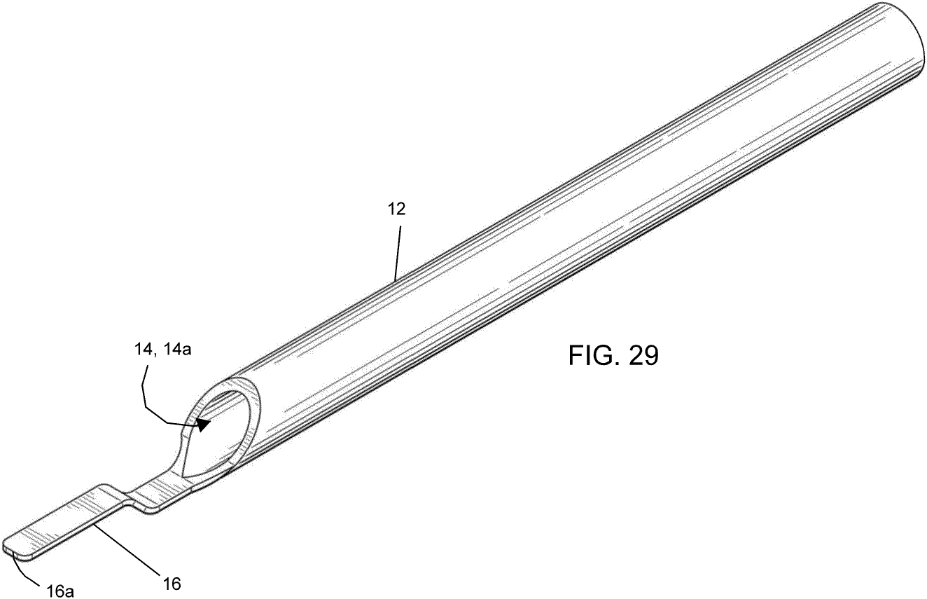

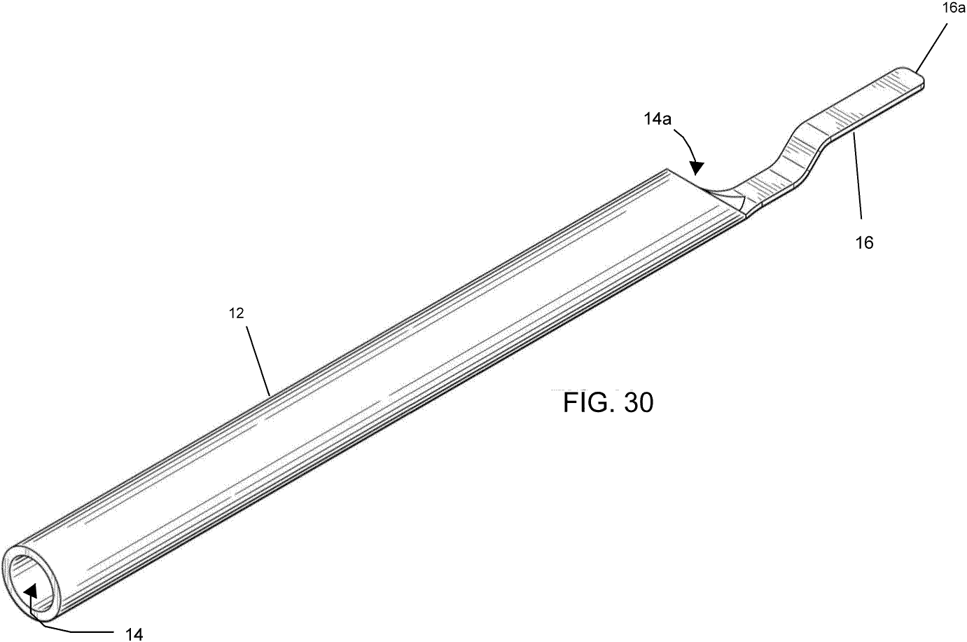

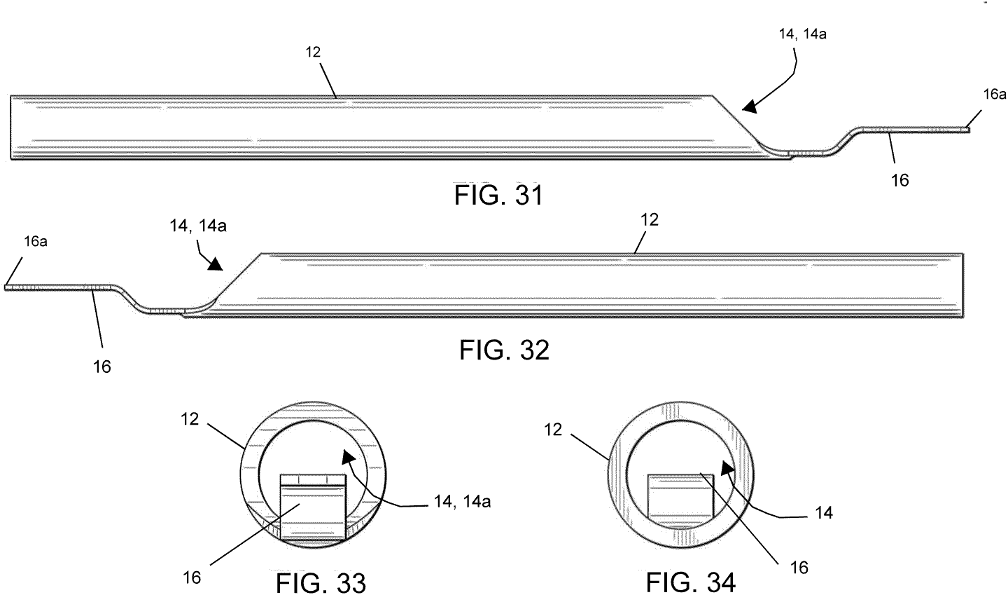

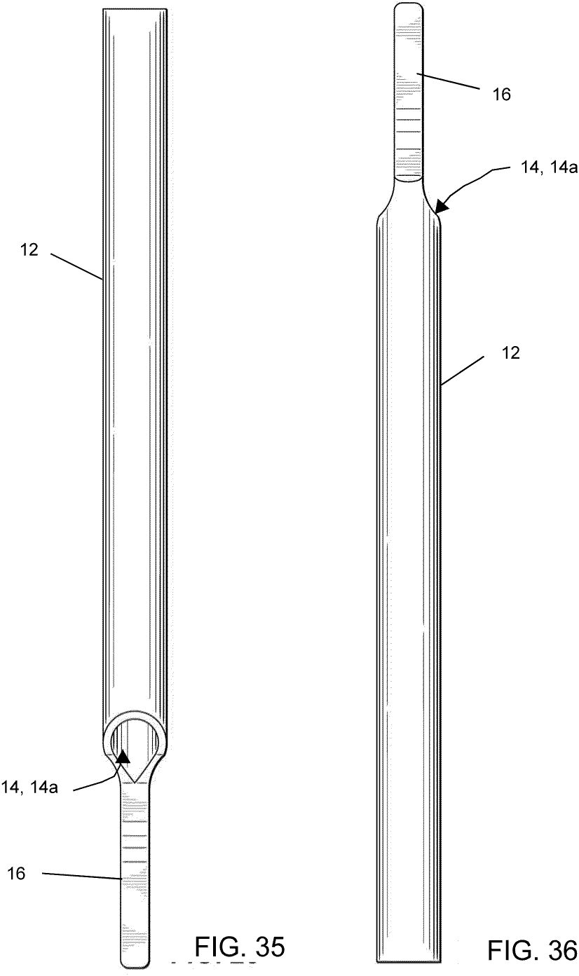

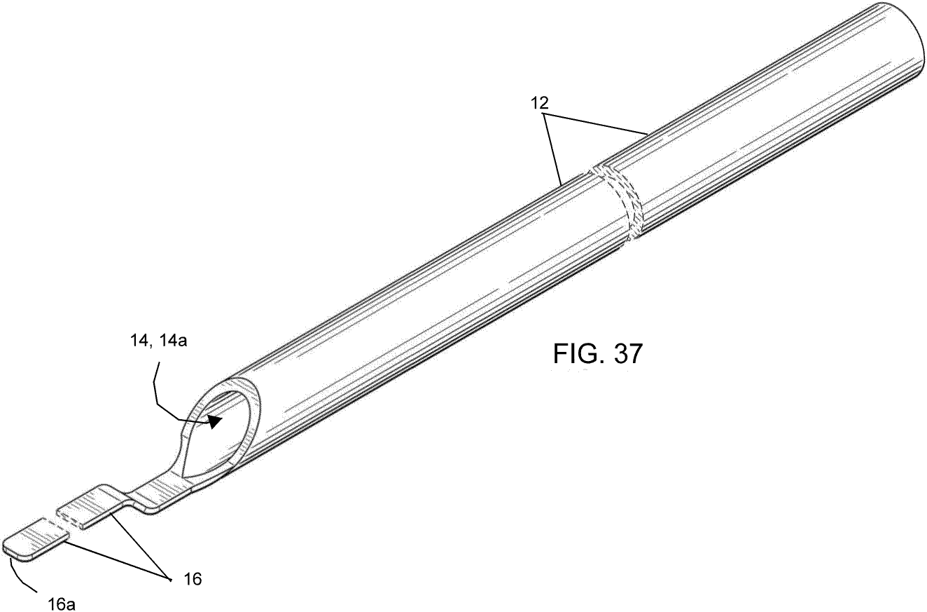

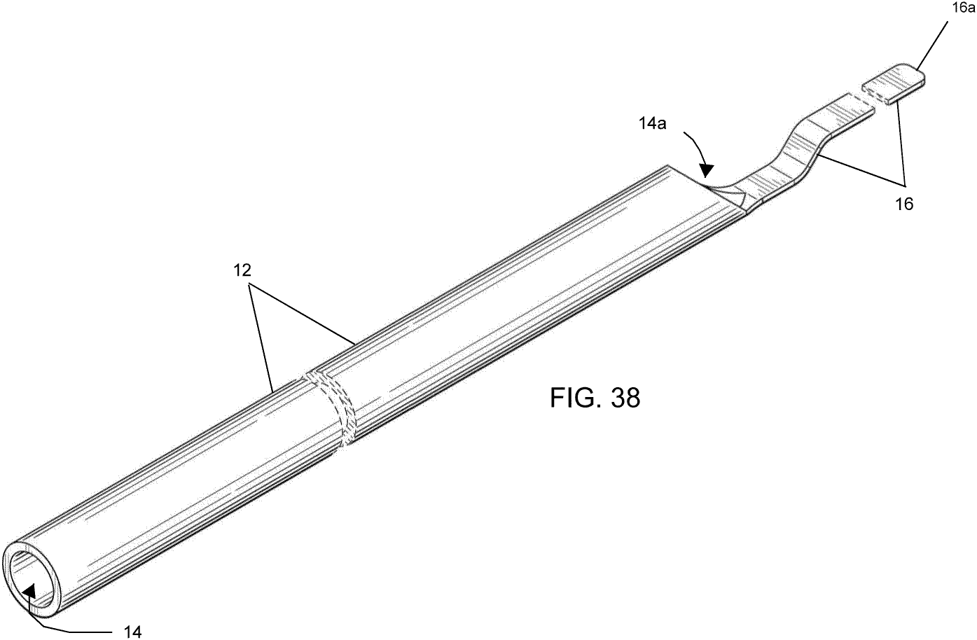

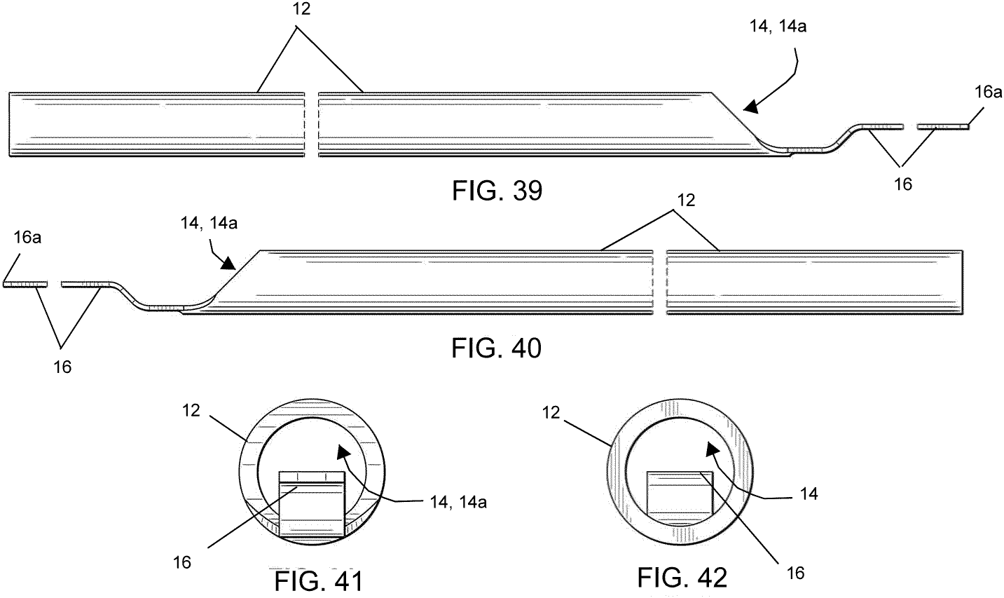

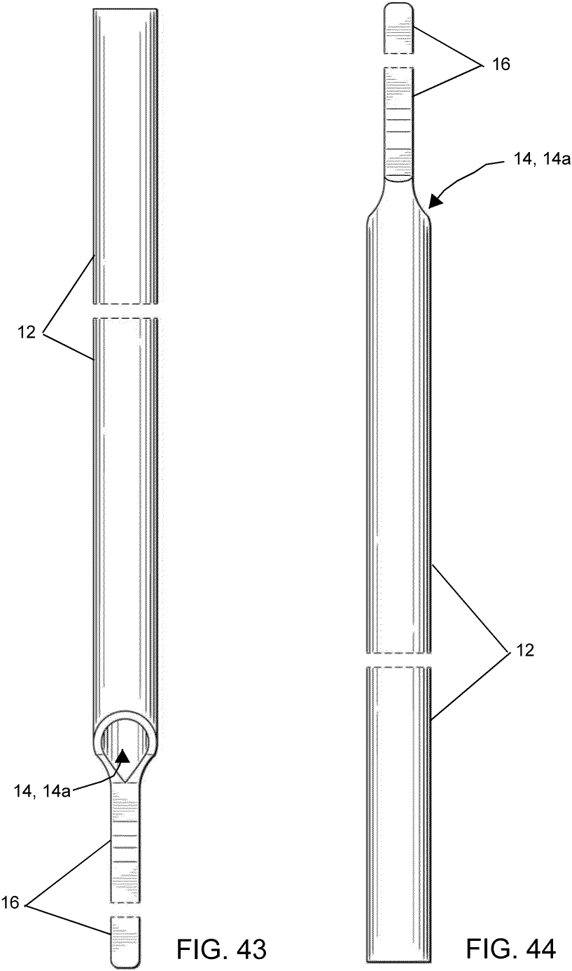

FIGS. 28-44 show the ornamental design of a bayonet cautery tip 16 depicted in FIGS. 6A-6I for use with the electrocautery instrument and system 10. FIG. 28 is a perspective view of a bayonet cautery tip as it is used with an electrocautery device. FIG. 29 is a right front perspective view of the cautery tip of FIG. 28. FIG. 30 is a left side rear perspective view of the cautery tip of FIG. 28. FIG. 31 is a left side view of the cautery tip of FIG. 1. FIG. 32 is a right side view of the cautery tip of FIG. 28. FIG. 33 is a front view of the cautery tip of FIG. 28. FIG. 34 is a rear view of the cautery tip of FIG. 28. FIG. 35 is a top view of the cautery tip of FIG. 28. FIG. 36 is a bottom view of the cautery tip of FIG. 28. FIG. 37 is a right front perspective view of the cautery tip of FIG. 28 shown with symbolic breaks in its length. FIG. 38 is left rear perspective view of the cautery tip of FIG. 28 shown with symbolic breaks in its length. FIG. 39 is a left side view of the cautery tip of FIG. 28 shown with symbolic breaks in its length. FIG. 40 is a right side view of the cautery tip of FIG. 28 shown with symbolic breaks in its length. FIG. 41 is a front view of the cautery tip of FIG. 28 shown with symbolic breaks in its length. FIG. 42 is a rear view of the cautery tip of FIG. 28 shown with symbolic breaks in its length. FIG. 43 is a top view of the cautery tip of FIG. 28 shown with symbolic breaks in its length. FIG. 44 is a bottom view of the cautery tip of FIG. 28 shown with symbolic breaks in its length.

### ADDITIONAL CONSIDERATIONS

1. Advantageously, the various tips 16 illustrated in FIGS. 2A-9E provide a plurality of embodiments with it being understood that in each embodiment, the cautery extension 19 and cautery tip 16 are integrally or monolithically formed with or conductively connected to the tubular body 12 as a result of machining the tubular body 12 and defines the generally centralized passageway or lumen 14 that passes through the entire electrocautery instrument and system 10.

2. Advantageously, the passageway or lumen 14 is generally linear and provides the conductive cautery tip 16 that is adapted for use during the cautery process. During the cautery procedure, the surgeon or user selects an appropriate electrocautery instrument and system 10 and cautery extension 19 having the desired shape of the cautery tip 16 for use during the procedure. The user sets a power level on the power supply 22 and energizes the electrocautery instrument and system 10 and cautery tip 16 by depressing the switch 20a to cause a current to flow into the cautery tip 16 so that the cauterization may be performed in a manner that is conventionally known. Both bipolar and monopolar approaches (FIGS. 10A-10B) may be used with these embodiments as mentioned earlier. It is contemplated that the electrocautery instrument and system 10 is adapted to use either an alternating current (AC) or a direct current (DC) power source or battery in a manner that is conventionally known.

3. FIGS. 9A-9E illustrate several different configurations of the cautery tip 16. For ease of illustration, the cautery tip 16 is shown, but it should be understood that each of the embodiments shown earlier may comprise the cautery tip 16 that comprises a custom or unique shape depending on the surgical procedure.

4. Providing the centralized passageway or lumen 14 through the electrocautery instrument and system 10 facilitates evacuating the smoke and vapor before the patient or personnel in the surgery room are exposed. The smoke and vapor may be evacuated into an ambient atmosphere, treated by the decontamination system or filtration device 20 and/or vented to a desired location, such as through a duct system or outside atmosphere.

5. The tubular body 12 may be provided to have a predetermined configuration for ease of handling. Although not shown, the tubular body 12 may have a shape that provides convenient grip areas on the tubular body 12 to facilitate gripping the instrument. As mentioned earlier, while it is shown to be generally cylindrical, it could have a different shape, such as polygonal, hexagonal or octagonal or the like. The surface 16d could also comprise ridges or other configurations to facilitate gripping the electrocautery instrument and system 10. The tubular body 12 and end wall 12b may or may not be insulated or overmolded with a coating or other insulation. For example, the tubular body 12 may be over-molded for providing a convenient grip or for aesthetic reasons.

6. The inner wall 12c defining the passageway or lumen 14 may also be coated with an insulative coating IC (FIG. 2I) for facilitating catalytic conversion of the undesirable smoke or vapor as described earlier. Several coating examples include materials which have oxidative catalyst properties, including elements or compounds containing platinum, palladium, manganese, copper, zirconium, redox-active oxides of iron, vanadium, and/or molybdenum.

7. It should be understood that the tubular body 12, the cautery extension 19 and the cautery tip 16 themselves provide the passageway or lumen 14 for smoke or vapor evacuation directly through the electrode or tubular body 12. Thus, the tubular body 12 defines a hollow electrode.

8. The design of the embodiments shown and described herein provide for a reduced-size instrument, improved surgical visualization and use and more clearance and a less bulky instrument for the surgeon to perform the cautery process. Given the centralized passageway or lumen 14 and its proximity to the cautery procedure, evacuation of unwanted smoke and vapor is improved when compared to the prior art.

Advantageously, another embodiment of this invention, including all embodiments shown and described herein, could be used alone or together and/or in combination with one or more of the features covered by one or more of the claims set forth herein, including but not limited to one or more of the features or steps mentioned in the Summary of the Invention and the claims.

While the system, apparatus and method herein described constitute preferred embodiments of this invention, it is to be understood that the invention is not limited to this precise system, apparatus and method, and that changes may be made therein without departing from the scope of the invention which is defined in the appended claims.

What is claimed is:

1. An electrocautery instrument for cauterizing a surgical site associated with a patient, said electrocautery instrument comprising:
a tubular body having a passageway for evacuating vapor from the surgical site;
said tubular body comprising an extension for cauterizing at the surgical site; said extension being conductive and adapted to be coupled to a power source for energizing said extension;
said tubular body having an inlet end having said extension and an outlet end coupled to a vacuum source adapted to create a predetermined pressure in said passageway in order to evacuate vapor from said surgical site through said tubular body prior to, during or after use of said electrocautery instrument at the surgical site, said vapor comprising at least one of smoke, cauterizing vapor, gas, liquid or debris; and
wherein said passageway is defined by said tubular body and causes said vapor resulting from the cauterizing at the surgical site to be evacuated through said tubular body.

2. The electrocautery instrument as recited in claim 1 wherein said tubular body is conductive and creates a conductive path for current to flow from a power source to said extension.

3. The electrocautery instrument as recited in claim 1 wherein said extension is integral with said tubular body and both are conductive.

4. The electrocautery instrument as recited in claim 3 wherein said tubular body is defined by a wall having at least one predetermined dimension, said wall defining said passageway and said extension.

5. The electrocautery instrument as recited in claim 4 wherein said extension comprising a cross-sectional dimension that is smaller than said at least one predetermined dimension.

6. The electrocautery instrument as recited in claim 4 wherein an elongated portion of said extension comprises a first surface and a generally opposing second surface, said first surface defining a working surface having a generally curved or arcuate portion.

7. The electrocautery instrument as recited in claim 6 wherein said working surface has a radius of curvature substantially approximating a radius of curvature of said wall.

8. The electrocautery instrument as recited in claim 6 wherein said extension comprises a working surface that is generally planar.

9. The electrocautery instrument as recited in claim 2 wherein said extension is detachably secured to said tubular body with a predetermined connection, said tubular body and said extension both being conductive to permit said extension to cauterize at said surgical site.

10. The electrocautery instrument as recited in claim 9 wherein said predetermined connection is a least one of a threaded, press-fit, bayonet, or socket connection.

11. The electrocautery instrument as recited in claim 1 wherein said passageway is substantially centrally located in said tubular body to define a substantially linear or non-serpentine conduit for evacuating said vapor.

12. The electrocautery instrument as recited in claim 1 wherein said tubular body comprises an interior wall that defines said passageway, at least a portion of said interior wall having a catalyst coating, a single uncoated material or other forms of deposition, such as plasma spraying and electroplating.

13. The electrocautery instrument as recited in claim 1 wherein said passageway is substantially centrally located in said tubular body to define a substantially linear or non-serpentine conduit for evacuating said vapor.

14. The electrocautery instrument as recited in claim 1 wherein extension comprises an elongated portion having a predetermined shape adapted to cauterize at the surgical site.

15. The electrocautery instrument as recited in claim 1 wherein said tubular body comprises an interior wall that defines said passageway to have a primary flow path through said tubular instrument, said extension lying lateral to said primary flow path.

16. The electrocautery instrument as recited in claim 1 wherein said tubular body comprises an interior wall that defines said passageway to have a primary flow path through said tubular instrument, said extension lying substantially in said primary flow path so that the vapor flows past at least a portion of said extension before it enters said passageway for evacuation.

17. The electrocautery instrument as recited in claim 16 wherein said extension comprises an extension axis that is generally coaxial with an axis of at least one of said tubular body or said passageway.

18. The electrocautery instrument as recited in claim 1 wherein said extension comprises an extension axis that is generally coaxial with an axis of at least one of said tubular body or said passageway.

19. The electrocautery instrument as recited in claim 4 wherein said extension comprises a first portion integrally formed with a first portion of said wall and having a first surface for performing work and a second surface for facilitating directing the vapor into said passageway.

20. The electrocautery instrument as recited in claim 4 wherein said extension comprises a first portion integrally formed in a first area of said wall and a second portion integrally formed in a second area of said wall, said extension comprising a first surface and generally opposed second surface, both of which are in an airflow path defined by said passageway.

21. The electrocautery instrument as recited in claim 20 wherein said first area and said second area are each associated with an inlet end of said tubular body, said extension comprising a cauterizing tip situated substantially between said first and second portions so that said first and second portions support said first and second surfaces in said airflow path to permit vapor to flow past said first and second surfaces and into said passageway.

22. The electrocautery instrument as recited in claim 21 wherein said extension is configured or adapted to facilitate directing and evacuating vapor into said passageway during a cauterizing procedure.

23. The electrocautery instrument as recited in claim 17 wherein said tubular body comprises an interior wall that defines said passageway to have a primary flow path through said tubular instrument, said extension lying lateral to said primary flow path.

24. The electrocautery instrument as recited in claim 9 wherein an elongated portion of said extension comprises a first surface and a generally opposing second surface, said first surface having a generally curved or arcuate portion.

25. The electrocautery instrument as recited in claim 9 wherein an elongated portion of said extension comprises a first surface and a generally opposing second surface, said first surface defining a working surface for engaging a patient and having a generally curved or arcuate portion.

26. The electrocautery instrument as recited in claim 25 wherein said first surface is generally curved and has a radius of curvature approximating a radius of curvature of an outer surface of said tubular body, said second generally opposing surface having a predetermined cross- sectional shape that is different from said first surface.

27. The electrocautery instrument as recited in claim 25 wherein said first surface is generally curved and said second surface is generally planar.

28. The electrocautery instrument as recited in claim 9 wherein an elongated portion of said extension comprises a first surface and a generally opposing second surface, said first and said second surfaces both being generally planar.

29. The electrocautery instrument as recited in claim 1 wherein said tubular body comprises an insulator.

30. The electrocautery instrument as recited in claim 20 wherein said tubular body comprises a non-conductive handpiece body for gripping said tubular body.

31. The electrocautery instrument as recited in claim 1 wherein said extension extends from an inlet end of said tubular body, said inlet end having an end wall portion that defines an inlet opening sized and adapted to received vapor from the surgical site.

32. The electrocautery instrument as recited in claim 31 wherein said end wall portion lies in a predetermined plane relative to an axis of said tubular body, said end wall portion of tubular body and said extension cooperating to define a predetermined angle of approximately 90 degrees or is acute.

33. The electrocautery instrument as recited in claim 32 wherein said end wall portion lies in a predetermined plane relative to an axis of said tubular body, said end wall portion defines an angled tubular body wall that lies in a plane relative to said axis of said tubular body that is obtuse.

34. The electrocautery instrument as recited in claim 1 wherein said tubular body comprises a working end having an end wall that lies in an imaginary plane that intersects an axis of said passageway at a predetermined angle.

35. The electrocautery instrument as recited in claim 34 wherein said predetermined angle is approximately 90 degrees.

36. The electrocautery instrument as recited in claim 34 wherein said predetermined angle is an angle other than 90 degrees.

37. The electrocautery instrument as recited in claim 1 wherein said extension is generally elongated and planar.

38. The electrocautery instrument as recited in claim 1 wherein said extension is generally non-planar.

39. The electrocautery instrument as recited in claim 38 wherein said extension is stepped and comprises a first surface that extends from a wall of said tubular body and a second surface, said first and second surfaces lying in different planes.

40. The electrocautery instrument as recited in claim 39 wherein said first and second surfaces are coupled by a joining portion, said first surface, said second surface and said joining portion being configured such that said second surface is situated substantially in an airflow path defined by said passageway.

41. The electrocautery instrument as recited in claim 40 wherein said second surface defines a cauterizing surface that is substantially centrally located in said airflow path, said airflow path through said tubular body being substantially linear.

42. An electrocautery handpiece which comprises:
a tubular handpiece body;
an internal airflow means;
a vacuum connection;
a substantially hollow tubular electrode communicating with said airflow means;
said electrode comprising an integral, substantially elongated extension from said tubular handpiece body, wherein electrical current is transmitted from said tubular handpiece body to said extension, and surgical smoke enters said tubular handpiece body and is evacuated via said internal airflow means.

43. The electrocautery handpiece of claim 42, wherein said surgical smoke arises from said electrode and is evacuated through said internal airflow means to said vacuum connection in a substantially linear manner.

44. The electrocautery handpiece of claim 42, wherein said internal airflow means comprises a channel located in the substantial center of an axis of said tubular handpiece body and is substantially linear.

45. The electrocautery handpiece of claim 42, wherein an evacuation airflow pathway travels in a substantially linear manner from its point of generation at the electrode to a vacuum connection means at a base of said handpiece.

46. The electrocautery handpiece of claim 42, wherein said electrocautery handpiece comprises said vacuum connection comprising a barbed fitting, threaded fitting, bonded fitting, swivel fitting, valve fitting or similar to allow for connection of said handpiece to a vacuum source via flexible surgical tube or similar implement.

47. The electrocautery handpiece of claim 42, wherein said electrocautery handpiece comprises an electrical supply and/or switching means for the provision of cauterizing current to said electrode, said current delivered to substantially tubular portion of said electrode for transmission to said extension.

48. An electrocautery electrode for the transmission of electrical current and surgical smoke plume, said electrode comprising a substantially tubular portion with at least one integral substantially linear extension;
said extension being integral with a portion of the tube wall.

49. The electrocautery electrode of claim 48, wherein said extension thickness is less than said tube wall thickness with at least one flattened surface opposing the curved surface of said tube wall.

50. The electrocautery electrode of claim 48, wherein a portion of said electrode and/or said extension is electrically insulated with a non-conductive coating, covering or material.

51. A method for the evacuation of surgical cautery smoke comprising generation of smoke plume from a substantially linear and/or flattened electrode tip, said tip being in integral communication with a tubular electrode body, said smoke flowing through a tubular electrode body in communication with an electrocautery handpiece comprising a substantially central lumen, said lumen being in communication with a vacuum source via a vacuum connection means to evacuate said smoke.
